SEQUENCE LISTING (7 sequences)

SEQ ID NO: 1 - protein, 10 aa, synthetic construct
MEQGEFLNYD

SEQ ID NO: 2 - protein, 15 aa, synthetic construct
QILMEQGEFLNYDIM

SEQ ID NO: 3 - protein, 20 aa, synthetic construct
DPQILMEQGEFLNYDIMLGV

SEQ ID NO: 4 - protein, 816 aa, Homo sapiens
MSRPQGLLWLPLLFTPVCVMLNSNVLLWLTALAIKFTLIDSQAQYPVVNTNYGKIRGLRTPLPNEILGPVEQYLGVPYASPPTGERRFQPPEPPSSWIGIRNTTQFAAVCPQHLDERSLLHDMLPIWFTANLDTLMTYVQDQNEDCLYLNIYVPTEDDIHDQNSKKPVMVYIHGGSYMEGTGNMIDGSILASYGNVIVITINYRLGILGFLSTGDQAAKGNYGLLDQIQALRWIEENVGAFGGDPKRVTIFGSGAGASCVSLLTLSHYSEGLFQKAIIQSGTALSSWAVNYQPAKYTRILADKVGCNMLDTTDMVECLRNKNYKELIQQTITPATYHIAFGPVIDGDVIPDDPQILMEQGEFLNYDIMLGVNQGEGLKFVDGIVDNEDGVTPNDFDFSVSNFVDNLYGYPEGKDTLRETIKFMYTDWADKENPETRRKTLVALFTDHQWVAPAVATADLHAQYGSPTYFYAFYHHCQSEMKPSWADSAHGDEVPYVFGIPMIGPTELFSCNFSKNDVMLSAVVMTYWINFAKTGDPNQPVPQDTKFIHTKPNRFEEVAWSKYNPKDQLYLHIGLKPRVRDHYRATKVAFWLELVPHLHNLNEIFQYVSTITKVPPPDMTSFPYGTRRSPAKIWPTTKRPAITPANNPKHSKDPHKTGPEDTTVLIETKRDYSTELSVTIAVGASLLFLNILAFAALYYKKDKRRHETHRRPSPQRNTTNDIAHIQNEEIMSLQMKQLEHDHECESLQAHDTLRLTCPPDYTLTLRRSPDDIPLMTPNTITMIPNTLTGMQPLHTFNTFSGGQNSTNLPHGHSTTRV

SEQ ID NO: 5 - protein, 1547 aa, Homo sapiens
MGTALLQRGGCFLLCLSLLLLGCWAELGSGLEFPGAEGQWTRFPKWNACCESEMSFQLKTRSARGLVLYFDDEGFCDFLELILTRGGRLQLSFSIFCAEPATLLADTPVNDGAWHSVRIRRQFRNTTLFIDQVEAKWVEVKSKRRDMTVFSGLFVGGLPPELRAAALKLTLASVREREPFKGWIRDVRVNSSQVLPVDSGEVKLDDEPPNSGGGSPCEAGEEGEGGVCLNGGVCSVVDDQAVCDCSRTGFRGKDCSQEIKFGLQCVLPVLLHDNDQGKYCCINTAKPLTEKDNNVEGLAHLMMGDQGKSKGKEEYIATFKGSEYFCYDLSQNPIQSSSDEITLSFKTLQRNGLMLHTGKSADYVNLALKNGAVSLVINLGSGAFEALVEPVNGKFNDNAWHDVKVTRNLRQHSGIGHAMVNKLHCSVTISVDGILTTTGYTQEDYTMLGSDDFFYVGGSPSTADLPGSPVSNNFMGCLKEVVYKNNDVRLELSRLAKQGDPKMKIHGVVAFKCENVATLDPITFETPESFISLPKWNAKKTGSISFDFRTTEPNGLILFSHGKPRHQKDAKHPQMIKVDFFAIEMLDGHLYLLLDMGSGTIKIKALLKKVNDGEWYHVDFQRDGRSGTISVNTLRTPYTAPGESEILDLDDELYLGGLPENKAGLVFPTEVWTALLNYGYVGCIRDLFIDGQSKDIRQMAEVQSTAGVKPSCSKETAKPCLSNPCKNNGMCRDGWNRYVCDCSGTGYLGRSCEREATVLSYDGSMFMKIQLPVVMHTEAEDVSLRFRSQRAYGILMATTSRDSADTLRLELDAGRVKLTVNLDCIRINCNSSKGPETLFAGYNLNDNEWHTVRVVRRGKSLKLTVDDQQAMTGQMAGDHTRLEFHNIETGIITERRYLSSVPSNFIGHLQSLTFNGMAYIDLCKNGDIDYCELNARFGFRNIIADPVTFKTKSSYVALATLQAYTSMHLFFQFKTTSLDGLILYNSGDGNDFIVVELVKGYLHYVFDLGNGANLIKGSSNKPLNDNQWHNVMISRDTSNLHTVKIDTKITTQITAGARNLDLKSDLYIGGVAKETYKSLPKLVHAKEGFQGCLASVDLNGRLPDLISDALFCNGQIERGCEGPSTTCQEDSCSNQGVCLQQWDGFSCDCSMTSFSGPLCNDPGITYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 6 - protein, 8 aa, synthetic construct
EQGEFLNY

SEQ ID NO: 7 - protein, 10 aa, synthetic construct
LEGEDQNFYM

(19) United States
(12) Patent Application Publication
(10) Pub. No.: US 2024/0409602 A1
(43) Pub. Date: Dec. 12, 2024

(54) PEPTIDE MODULATORS OF NEUROLIGIN 4-NEUREXIN 1-BETA AXIS FOR TREATMENT OF LIVER DISORDERS

(71) Applicant: HADASIT MEDICAL RESEARCH SERVICES AND DEVELOPMENT LTD., Jerusalem (IL)

(72) Inventors: Rifaat SAFADI, Jerusalem (IL); Johnny AMER, Jerusalem (IL)

(21) Appl. No.: 18/812,184

(22) Filed: Aug. 22, 2024

Related U.S. Application Data
(63) Continuation of application No. PCT/IL2023/050231, filed on Mar. 6, 2023.
(60) Provisional application No. 63/317,237, filed on Mar. 7, 2022.

(51) Int. Cl. C07K 14/705; A61K 35/17; A61K 38/00; A61P 1/16; A61P 35/00; C12N 5/0783

(52) U.S. Cl. CPC C07K 14/705; A61K 35/17; A61P 1/16; A61P 35/00; C12N 5/0646; A61K 38/00

(57) ABSTRACT

Provided herein are isolated peptides capable of affecting neuroligin-4 (NLGn4)-neurexin 1β (Nrx1β) protein-protein interaction and interfering with NLGn4X/Nrx1β axis between Hepatic Stellate Cells (HSCs) and Natural killer (NK). Further provided are compositions including such peptides and uses thereof for treating and/or attenuating liver-related conditions and various types of cancer.

### CROSS REFERENCE TO RELATED APPLICATIONS

This application is a bypass continuation of PCT/IL2023/050231 filed Mar. 6, 2023, which claims the benefit of priority of U.S. Provisional Patent Application No. 63/317,237, Mar. 7, 2022, entitled “PEPTIDE MODULATORS OF NEUROLIGIN 4-NEUREXIN 1-BETA AXIS FOR TREATMENT OF LIVER DISORDERS.” The contents of both applications are incorporated herein by reference in their entireties.

### REFERENCE TO AN ELECTRONIC SEQUENCE LISTING

The contents of the electronic sequence listing (HDST_061-PCT.xml; size: 9,584 bytes; and date of creation: Aug. 22, 2024) is herein incorporated by reference in its entirety.

### FIELD OF THE INVENTION

The present invention relates to novel peptides capable of affecting neuroligin-4 (NLGn4)-neurexin 1β (Nrx1β) protein-protein interactions, compositions including the same and uses thereof in treating and/or attenuating liver-related conditions and various types of cancer.

### BACKGROUND OF THE INVENTION

The liver is an intricate organ, composed of hepatocytes and non-parenchymal cells, which includes, Kupffer cells, endothelial cells, myofibroblasts known as Hepatic Stellate Cells (HSCs) and leukocytes. In healthy livers, HSCs are in a quiescent state, but when the liver is chronically injured, HSCs are transformed into an activated state characterized by enhanced proliferation and production of excessive extracellular matrix (ECM), as part of the pathogenesis of liver fibrosis.

Natural killer (NK) cells play a critical role in the early stages of the immune innate response. NK cells participate in the process of liver fibrosis and serve as anti-fibrotic activity through HSCs killing, but their function decreases when the liver disease progresses into cirrhosis in a long-standing disease.

Neuroligin-4 (NLG4, NLGn4, NLGn4X) is a family member of neuronal cell surface proteins called the Neuroligins. Members of this family are membrane-anchored proteins acting as ligands for beta-neurexins. Neurexin (NRXN), namely neurexin 1β (Nrx1β), is a member of the presynaptic protein family (Neuroligins ligands) that help to glue together neurons at the synapse. Neurexins are located mostly on the presynaptic membrane and contain a single transmembrane domain. The extracellular domain interacts with proteins in the synaptic cleft, most notably neuroligin, while the intracellular cytoplasmic portion interacts with proteins associated with exocytosis.

The NLGn4X/Nrx1β axis has been shown to be related to liver physiology and pathology. Prolonged stimulation of the NLGn4X/Nrx1β correlates with the progression of liver fibrosis. An interplay that contributes to the progression of liver fibrosis, is considered to involve the NLGn4X/Nrx1β axis between HSCs and NK cells.

International application, publication No. WO 2016/157195 to some of the inventors of the present application is directed to antibodies and recombinant proteins capable of interfering with, inhibiting and/or preventing NLGn4-Nrx1β protein-protein interactions (PPI), and to the use thereof for the treatment and/or attenuation of liver disorders.

Nevertheless, there is an unmet need in the art for improved compositions, which are cost effective, safe and efficient, with reduced side effects, for use in treating liver related conditions, by affecting the NLGn4-Nrx1p interaction in various cells and tissues.

### SUMMARY OF THE INVENTION

According to some embodiments, provided herein are advantageous isolated inhibitory peptides, capable of effectively and safely affecting NLGn4X-Nrx1β interactions, as well as compositions including the same, for treating various liver-related conditions, such as, fibrosis, non-alcoholic fatty liver disease (NAFLD), non-alcoholic steatohepatitis (NASH), cirrhosis, hepatitis, liver adenoma, insulin hypersensitivity, liver cancer and any combination thereof.

According to some embodiments, the present invention stems, in part, from the surprising finding that specific isolated peptides capable of affecting (for example, by interfering or inhibiting) NLGn4X-Nrx1β protein-protein interactions can modulate the NLGn4X-Nrx1β axis in a manner that crucially disrupts functional intercellular attributes of, for example, hepatic satellite cells (HSCs) or liver cancer cells, which play an essential role in the progression of various liver disorders. In some embodiments, the specific peptides are directed against NLGn4X and/or Nrx1β, thereby exerting a biological effect.

According to some embodiments, without wishing to be bound by any theory or mechanism, it was surprisingly found that pre-activation of NK cells with inhibitory peptides of the invention, can synergistically deactivate HSCs, thereby treating or ameliorating related liver conditions.

According to some embodiments, without wishing to be bound by any theory or mechanism, it was surprisingly found that hepatocellular cells (HCC) treated with the inhibitory peptides disclosed herein, proliferate/divide at slower rate and/or more prone for programed cell death/apoptosis. Accordingly, the peptides disclosed herein can be used for treating or ameliorating liver related conditions, such as, liver cancer.

According to further embodiments, without wishing to be bound by any theory or mechanism, it was surprisingly found that the NLGn4-Nrx1β inhibitory peptides can advantageously promote anti-fibrotic effects, by functionally reducing hepatic stellate cell activation-mediated phagocytosis and/or increasing NK cells adherence-mediated killing of hepatic stellate cells (HSCs).

According to some embodiments, there is provided an isolated modulator peptide having an amino acid sequence as denoted by SEQ ID NO: 6, wherein the isolated peptide is capable of affecting interactions between neuroligin 4 (NLGn4X) and Neurexin 1β (Nrx1β).

According to some embodiments, the isolated modulator peptide has an amino acid sequence as denoted by any one of SEQ ID NO: 1, SEQ ID NO: 2 and SEQ ID NO: 3.

According to some embodiments, the isolated modulator peptide consists of an amino acid sequence as denoted by any one of SEQ ID NO: 1, SEQ ID NO: 2, SEQ ID NO: 3 and/or SEQ ID NO: 6.

According to some embodiments, there is provided a composition which includes the isolated modulator peptide(s), and a pharmaceutically acceptable carrier.

According to some embodiments, the composition may include a plurality of isolated modulator peptides.

According to some embodiments, the isolated modulator peptide(s), or the composition including the same may be used for treating, attenuating, and/or preventing progression of a liver disorder, in a subject in need thereof.

According to some embodiments, the liver disorder may be selected from: fibrosis, non-alcoholic fatty liver disease (NAFLD), non-alcoholic steatohepatitis (NASH), cirrhosis, hepatitis, viral hepatitis, liver adenoma, insulin hypersensitivity, liver cancer, cholangiocarcinoma, liver metastasis or any combination thereof.

According to some embodiments, the liver disorder is liver fibrosis or liver cancer. Each possibility is a separate embodiment.

According to some embodiments, the isolated modulator peptide(s), or the composition including the same may be used for treating, attenuating, and/or preventing progression of a cancer condition in a subject in need thereof. In some embodiments, the cancer may be selected from: liver cancer, colorectal cancer, breast cancer and prostate cancer.

According to some embodiments, the isolated modulator peptide or the composition may be administered systematically.

According to some embodiments, the isolated modulator peptide or the composition may be administered in combination with at least one additional therapeutic agent.

According to some embodiments, the isolated modulator peptide, or the composition including the same may be used for reducing activation of hepatic stellate cells (HSCs).

According to some embodiments, reducing activation of hepatic stellate cells (HSCs) comprises increased susceptibility of killing thereof by activated natural killer (NK) cells and/or reduced engulfment by activated NK cells.

According to some embodiments, the isolated modulator peptide or the composition including the same may be used for attenuating proliferation and/or oncogenicity of cancer cells, such as, liver cancer cells, colorectal cancer cells, breast cancer cells and/or prostate cancer cells.

According to some embodiments, the attenuation of proliferation and/or oncogenicity of cancer cells may be associated with reduced entry of the cancer cells into a state of existing in S-phase or G2-M phase of a cell cycle and increased entry of the cancer cells into a state of existing in G1-phase.

According to some embodiments, the attenuation of proliferation and/or oncogenicity of cancer cells is associated with an increase in programmed cell death/apoptosis of the cancer cells and/or reduction in necrosis of the cancer cells.

According to some embodiments, reducing activation of hepatic stellate cells (HSCs) comprises increased susceptibility for killing thereof by natural killer (NK) cells.

According to some embodiments, there is provided a method of treating, attenuating and/or preventing progression of a liver disorder in a subject in need thereof, the method including administering a therapeutically effective amount of the modulator peptide(s) or a composition including the same. According to some embodiments, the liver disorder may be selected from fibrosis, non-alcoholic fatty liver disease (NAFLD), non-alcoholic steatohepatitis (NASH), cirrhosis, hepatitis, liver adenoma, insulin hypersensitivity, liver cancer.

According to some embodiments, the administration may include systemic or localized administration.

According to some embodiments, the method may further include administering at least one additional therapeutic agent.

According to some embodiments, there is provided a natural killer (NK) cell harboring or expressing the isolated modulator peptide or composition including the same.

According to some embodiments, the isolated peptide is introduced or penetrates the NK cell as a peptide. According to another embodiment, the isolated peptide is introduced into the NK cells as a nucleic acid encoding the same.

According to some embodiments, the NK cell is an activated cell capable of reducing or inhibiting activity of hepatic stellate cells.

According to some embodiments, there is provided a composition comprising the NK cell harboring or expressing the isolated modulator peptide or composition including the same.

According to some embodiments, the NK cell harboring or expressing the isolated modulator peptide or composition including the same is for use in treating, attenuating and/or preventing progression of a liver disorder in a subject in need thereof.

According to some embodiments, the NK cell harboring or expressing the isolated modulator peptide or composition including the same is for use in treating, attenuating and/or preventing progression of cancer condition in a subject in need thereof.

According to some embodiments, the NK harboring or expressing the isolated modulator peptide or composition including the same is administered systematically.

According to some embodiments, the NK harboring or expressing the isolated modulator peptide or composition including the same is administered in combination with at least one additional therapeutic agent.

According to some embodiments, reduction in activity of HSC by NK-cells harboring the isolated modulator peptide or the composition comprising the same is synergistic, with respect to reduction in activity of HSC by NK-cells not harboring the isolated modulator peptide or the composition comprising the same, and with respect to reduction in activity of HSC by the isolated modulator peptide or the composition comprising the same.

According to some embodiments, the NK-cells harboring the isolated modulator peptide the composition comprising the same, having reduced susceptibility of being engulfed into HSCs, and/or increased functionality in killing HSCs, and/or increased attachment/adherence to HSCs. Each possibility is a separate embodiment.

According to some embodiments, there is provided a method of treating, attenuating and/or preventing progression of a liver disorder in a subject in need thereof, the method includes administering a therapeutically effective amount of the modulator peptide or a composition including the same.

According to some embodiments, there is provided a method of treating, attenuating and/or preventing progression of a liver disorder in a subject in need thereof, the method comprising administering a therapeutically effective amount of the NK cell harboring the modulator peptide and/or the composition including the NK cell.

According to some embodiments, there is provided a method of treating, attenuating and/or preventing progression of cancer condition in a subject in need thereof, the method includes administering a therapeutically effective amount of one or more of: an NK cell harboring the modulator peptide, composition including the NK cell, the modulator peptide(s), and/or composition including the modulator peptide(s).

According to some embodiments, the liver disorder is selected from fibrosis, non-alcoholic fatty liver disease (NAFLD), non-alcoholic steatohepatitis (NASH), cirrhosis, hepatitis, liver adenoma, insulin hypersensitivity, liver cancer. Each possibility is a separate embodiment.

According to some embodiments, the liver disorder is liver fibrosis. According to some embodiments, the liver disorder is liver cancer.

According to some embodiments, the cancer may be selected from, but not limited to: breast cancer, liver cancer, colorectal cancer and/or prostate cancer.

According to some embodiments, the administering may include systemic or localized administration.

According to some embodiments, the method may further include administering at least one additional therapeutic agent.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 presents a graph showing NK cells activation utilizing isolated peptide 1. CD107a levels in NK cells in monoculture were measured by flow cytometry, in the presence or absence of isolated peptide 1. The results are presented as % change from non-treated NK cells relative to maximal activation. NK cells were stimulated with different concentrations of 2000 ng/ml, 4000 ng/ml, and 8000 ng/ml of peptide 1 (NLGn4-Nrx1β inhibitory peptide 1) and compared to untreated (NT) cells. (ns, *, **, *** indicates p>0.05, p=<0.05, p=<0.01, p=<0.001, respectively, as calculated from six technical repetitions). The dotted line represents the upper normal limit for NK cell activation in a healthy situation;

FIGS. 2A-2B present graphs showing NK cells activation utilizing isolated peptides. CD107a levels in peptide-treated or non-treated NK cells in monoculture, measured by flow cytometry, and presented as % change from non-treated NK cells relative to maximal activation (FIG. 2A) and as % increase in change relative to untreated cells (FIG. 2B). NK cells were stimulated with different concentrations of 2000 ng/ml, 4000 ng/ml, and 8000 ng/ml of peptide 1 or peptide 2, or 2000 ng/ml or 4000 ng/ml of peptide 3, and compared to untreated cells (NT). (ns, *, **, *** indicates p>0.05, p=<0.05, p=<0.01, p=<0.001, respectively, as calculated from six technical repetitions). The dotted line represents the upper normal limit for NK cell activation in a healthy situation;

FIGS. 3A-3C show graphs of aSMA (Alpha Smooth Muscle Actin) levels in LX2 cells in monoculture, measured by flow cytometry, and presented as change in mean fluorescence intensity (MFI) relative to untreated cells (FIG. 3A), as % decrease in change relative to untreated cells (FIG. 3B), and as change in mean fluorescence intensity (MFI) relative to untreated cells (FIG. 3C). LX2 cells were stimulated with different concentrations of 2000 ng/ml, 4000 ng/ml, and 8000 ng/ml of NLGn4-Nrx1β peptide 1 or peptide 2, or 2000 ng/ml or 4000 ng/ml of peptide 3, and compared to untreated cells (NT). (ns, *, **, *** indicates p>0.05, p=<0.05, p=<0.01, p=<0.001, respectively, as calculated from six technical repetitions). The dotted line represents the averages obtained across all experiments for LX2 activations without treatments with peptide 1;

FIGS. 4A-4C show graphs of aSMA (Alpha Smooth Muscle Actin) levels in LX2 cells in co-culture with stimulated NK cells, measured by flow cytometry, and presented as change in mean fluorescence intensity (MFI) relative to untreated co-cultured cells (FIG. 4A), as % decrease in change relative to untreated co-cultured cells (NT) (FIG. 4B) and as change in mean fluorescence intensity (MFI) relative to untreated co-cultured cells (FIG. 4C). NK cells were pre-activated with different concentrations of 2000 ng/ml, 4000 ng/ml, and 8000 ng/ml of peptide 1 or peptide 2 or 2000 ng/ml or 4000 ng/ml of peptide 3, and compared to untreated co-cultured cells (NT) (ns, *, **, *** indicates p>0.05, p=<0.05, p=<0.01, p=<0.001, respectively, as calculated from six technical repetitions). The dotted line represents the averages obtained across all experiments for LX2 activations without treatments with peptide 1;

FIGS. 5A-5B show graphs of NK cells co-cultured with LX2 cells (CD56/αSMA). FIG. 5A shows highly activated cells represented as αSMA+ with 52% activation (represented as dot line). Pre-stimulated NK cells (CD56+ cells) with peptide 1 deactivated these cells. The more active NK cells correlate with more killed LX2 cells FIG. 5B shows less activated LX2 co-cultured with peptide 1 treated-NK cells represented as (CD56+/αSMA−). The data indicate that deactivated LX2 cell population presented in FIG. 5A is shifted to be αSMA− in FIG. 5B and NK cells show less cytotoxicity to kill LX2 because of their quiescent status. Before co-culture with LX2 cells, NK cells were pre-activated with different concentrations of 2000 ng/ml, 4000 ng/ml, and 8000 ng/ml of peptide 1 and compared to untreated co-cultured cells (NT). (ns, *, **, *** indicates p>0.05, p=<0.05, p=<0.01, p=<0.001, respectively, as calculated from six technical repetitions). Results indicate a necessary activation condition of LX2 for them to be deactivated through the peptides influenced-NK cells;

FIGS. 6A-6D show line graphs of weekly measurements of several parameters related to mice appetite, liver toxicity, and body weight, presented as the average change in weight (FIG. 6A), food intake (FIG. 6B), water intake (FIG. 6C), and liver weight (FIG. 6D), compared between naïve mice (marked as 0 mg/mice) and CCl₄-induced mice (acute hepatic fibrosis model) which were treated tri-weekly (3×/week) for 4 weeks with two doses (5 mg/mice or 10 mg/mice) of either a scrambled peptide (left hand panel) or the NLGn4-Nrx1β PPI inhibitory peptide 1 (right hand panel). (ns, *, **, *** indicates p>0.05, p=<0.05, p=<0.01, p=<0.001, respectively);

FIG. 7A shows bar graphs of analyses of the changes in serum levels of alanine transaminase ALT measured by ELISA and presented as fold changes; and

FIGS. 7B-7D show pictograms of the changes in liver pathology assessed using different histological staining, H&M (FIG. 7B), Sirius Red (FIG. 7C), and Masson's trichrome (FIG. 7D), compared between naïve mice and CC14-induced mice (acute hepatic fibrosis model) which were treated tri-weekly (3×/week) for 4 weeks with two doses (5 mg/mice or 10 mg/mice) of either a scrambled peptide (“SCR”) or the NLGn4-Nrx1β peptide 1. (ns, *, **, *** indicates p>0.05, p=<0.05, p=<0.01, p=<0.001, respectively);

FIGS. 8A-8E show analyses of the changes in expression levels of a panel of fibrotic markers expressed in the liver including αSMA mRNA (FIG. 8A) and protein (FIG. 8B), collagen I mRNA (FIG. 8C), CREBP mRNA (FIG. 8D), and MMP-9 protein (FIG. 8E), measured by RT-PCR and western blot and presented as fold changes, compared between naïve mice and CC14-induced mice (acute hepatic fibrosis model) which were treated tri-weekly (3×/week) for 4 weeks with two doses (5 mg/mice or 10 mg/mice) of either a scrambled peptide or peptide 1. (ns, *, **, *** indicates p>0.05, p=<0.05, p=<0.01, p=<0.001, respectively);

FIG. 9 shows analyses of the changes in expression levels of β-neurexin expressed in activated liver HSCs (shown in FIG. 10E) measured by RT-PCR and presented as fold changes, compared between naïve mice and CC14-induced mice (acute hepatic fibrosis model) which were treated tri-weekly (3×/week) for 4 weeks with two doses (5 mg/mice or 10 mg/mice) of either a scrambled peptide or the NLGn4-Nrx1p inhibitory peptide 1. (ns, *, **, *** indicates p>0.05, p=<0.05, p=<0.01, p=<0.001, respectively);

FIGS. 10A-10B show analyses of the changes in expression levels of F-actin expressed in isolated liver NK cells, measured by western blot (FIG. 10A) and quantified bar graphs (FIG. 10B), compared between naïve mice and CCl₄-induced mice (acute hepatic fibrosis model) which were treated tri-weekly (3×/week) for 4 weeks with two doses (5 mg/mice or 10 mg/mice) of either a scrambled peptide (“scr”) or the NLGn4-Nrx1β inhibitory peptide 1. (ns, *, **, *** indicates p>0.05, p=<0.05, p=<0.01, p=<0.001, respectively); and

FIGS. 11A-11B show representative immunofluorescence (IF) image analyses of the changes in the expression and localization of F-actin inside isolated liver NK cells, as detected by confocal microscopy. F-actin localization to the cells was assessed between CCl₄-induced mice treated with NLGn4-Nrx1β PPI modulator peptide 1 (FIG. 11A; ×25 zoom) to CCl₄-induced mice treated with a scrambled peptide (FIG. 11B; ×40 zoom), by comparing overlapping images of cells stained against DAPI (upper left), Natural Cytotoxicity Receptor NKP46 (NK1.1, upper right), F-actin (bottom left), and merge (bottom right).

FIGS. 12A-12B shows analyses of β-neurexin expression levels in the Hepatocellular carcinoma (HCC) cell line Hep3B as measured by western blot (FIG. 12A) or flow cytometry (FIG. 12B).

FIGS. 13A-13D show bar graphs of gene expression analyses of proliferation and oncogenic markers in the Hepatocellular carcinoma (HCC) cell line Hep3B treated with 8000 ng/ml of peptide 1 or scrambled control peptide. Expression of α-feto-protein (α-FP) was measured by ELISA (FIG. 13A), and of Carboxy Fluorescein Succinimidyl Ester (CSFE) (FIG. 13B), PDGFRA (FIG. 13C) and MKI67 (FIG. 13D) was measured by flow cytometry. (ns, *, **, *** indicates p>0.05, p=<0.05, p=<0.01, p=<0.001, respectively, as calculated from six technical repetitions).

FIGS. 14A-14C show representative histograms of flow cytometry analysis of Hepatocellular carcinoma (HCC) cells of the Hep3B cell line treated with peptide 1 (FIG. 14A) or scrambled control peptide (FIG. 14B), and sorted according to their size (forward scatter) and granularity (side scatter), and including viable cells (Gate 1) (FIG. 14C). Cells were stained with Propidium Iodide (PI) for fragmented DNA.

FIGS. 15A-15C show violine plots of flow cytometry analysis of Hepatocellular carcinoma (HCC) cells of the Hep3B cell line treated with 8000 ng/ml of peptide 1 or scrambled control peptide. Expression analysis of phosphorylated markers of the Akt- and mTOR (mammalian target of rapamycin)—signaling pathways included expression of phospho-AKT (S 473) (FIG. 15A), phospho-mTOR (S 2448) (FIG. 15B), and phospho-P70S6K (S 473) (FIG. 15C). (ns, *, **, *** indicates p>0.05, p=<0.05, p=<0.01, p=<0.001, respectively, as calculated from six technical repetitions.

FIGS. 16A-16C show bar graphs of flow cytometry analysis of Hepatocellular carcinoma (HCC) cells of the Hep3B cell line treated with 8000 ng/ml of peptide 1 or scrambled control peptide. Gene expression analysis of cellular markers for cell death—necrosis or apoptosis—included determining levels of expression of the necrotic marker CDKN2A (FIG. 16A) or the apoptotic marker phosphatidylserine which is represented as Annexin-V+/PI− for apoptotic cells (FIG. 16B) and as Annexin-V−/PI+ for viable cells (FIG. 16C). Cells were stained with Propidium Iodide (PI) for fragmented DNA. (ns, *, **, *** indicates p>0.05, p=<0.05, p=<0.01, p=<0.001, respectively, as calculated from six technical repetitions).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

### Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The following are terms that are used throughout the description and which should be understood in accordance with the various embodiments to mean as follows:

As used herein, the terms “isolated peptide”, “modulator peptide”, “inhibitory peptide”, “NLGn4-Nrx1β protein-protein interaction (PPI) inhibitor peptide”, “NLGn4-Nrx1β protein-protein interaction (PPI) modulator peptide” and “NLGn4-Nrx1β inhibitory peptide” may be used interchangeably. The terms relate to an isolated peptide (and/or compositions including the same) that may interact with and/or bind to a corresponding site/region in NLGn4 and/or Nrx1β, to thereby modulate and/or interfere with the interaction (for example, protein-protein interaction) between NLGn4 and Nrx1β. In some embodiments, the binding and/or modulation/interference is transient. In some embodiments, the binding and/or modulation/interference is reversible. In some embodiments, the binding and/or modulation/interference is permanent (irreversible). The binding of the inhibitory peptide to NLGn4 and/or Nrx1p may occur both in-vivo or in-vitro. For example, in some embodiments, binding of the inhibitory peptide to NLGn4 and/or Nrx1β may occur in-vivo, in tissue/cells of a subject (for example, NK cells). For example, in some embodiments, binding of the inhibitory peptide to NLGn4 and/or Nrx1β may occur in-vitro, i.e., the inhibitory peptide may bind NLGn4/Nrx1β in NK cells extracted from the subject's body. In some embodiments, such NK cells may later be administered to the patient.

As used herein, the terms “modulate”, “affect”, “alter” and “interfere”, may be used interchangeably. The phrase “capable of affecting/modulating/interfering” as used herein refers to the capability of an isolated peptide to inhibit or prevent interaction or binding between NLGn4 and Nrx1β or to interact with a target of NLGn4 or Nrx1β, such that the target becomes less accessible, preferably inaccessible, to binding by its corresponding binding partner (for example, Nrx1β or NLGn4), for example the receptor's natural antigen. In some embodiments, the isolated peptide is capable of inhibiting binding between NLGn4 and Nrx1β.

The term “binding site” or “epitope” as used herein refers to the region of NLGn4 and/or Nrx1β that specifically reacts with a particular modulator peptide.

The term “activated NK cells” relates to NK cells treated (administered, expressing, introduced with) an isolated peptide of the invention, or a composition including the same.

The terms “Neuroligin 4”, “Neuroligin 4X”, “NLGn4” and “NLG4” are interchangeable and as used herein refer to the protein product of the NLGn4 gene e.g., NP_001269075.1, NP_001269074.1, NP_851849.1 and NP_065793.1.

In some embodiments, the amino acid sequence of NLGn4 protein is denoted by SEQ ID NO: 4:

MSRPQGLLWLPLLFTPVCVMLNSNVLLWLTALAIKFTLIDSQAQYPVVN
TNYGKIRGLRTPLPNEILGPVEQYLGVPYASPPTGERRFQPPEPPSSWI
GIRNTTQFAAVCPQHLDERSLLHDMLPIWFTANLDTLMTYVQDQNEDCL
YLNIYVPTEDDIHDQNSKKPVMVYIHGGSYMEGTGNMIDGSILASYGNV
IVITINYRLGILGFLSTGDQAAKGNYGLLDQIQALRWIEENVGAFGGDP
KRVTIFGSGAGASCVSLLTLSHYSEGLFQKAIIQSGTALSSWAVNYQPA
KYTRILADKVGCNMLDTTDMVECLRNKNYKELIQQTITPATYHIAFGPV
IDGDVIPDDPQILMEQGEFLNYDIMLGVNQGEGLKFVDGIVDNEDGVTP
NDFDFSVSNFVDNLYGYPEGKDTLRETIKFMYTDWADKENPETRRKTLV
ALFTDHQWVAPAVATADLHAQYGSPTYFYAFYHHCQSEMKPSWADSAHG
DEVPYVFGIPMIGPTELFSCNFSKNDVMLSAVVMTYWINFAKTGDPNQP
VPQDTKFIHTKPNRFEEVAWSKYNPKDQLYLHIGLKPRVRDHYRATKVA
FWLELVPHLHNLNEIFQYVSTITKVPPPDMTSFPYGTRRSPAKIWPTTK
RPAITPANNPKHSKDPHKTGPEDTTVLIETKRDYSTELSVTIAVGASLL
FLNILAFAALYYKKDKRRHETHRRPSPQRNTINDIAHIQNEEIMSLQMK
QLEHDHECESLQAHDTLRLTCPPDYTLTLRRSPDDIPLMTPNTITMIPN
TLTGMQPLHTFNTFSGGQNSTNLPHGHSTTRV

The terms “neurexin-1-beta”, “β-neurexin” “Nrx1β” and “Nrxn1” are interchangeable and as used herein refer to the protein product of the Nrx1β gene e.g., NP_001129131.1 NP_004792.1 NP_620072.1. In some embodiments, the amino acid sequence of Nrx1β protein is denoted by SEQ ID NO: 5:

MGTALLQRGGCFLLCLSLLLLGCWAELGSGLEFPGAEGQWTRFPKWNAC
CESEMSFQLKTRSARGLVLYFDDEGFCDFLELILTRGGRLQLSESIFCA
EPATLLADTPVNDGAWHSVRIRRQFRNTTLFIDQVEAKWVEVKSKRRDM
TVFSGLFVGGLPPELRAAALKLTLASVREREPFKGWIRDVRVNSSQVLP
VDSGEVKLDDEPPNSGGGSPCEAGEEGEGGVCLNGGVCSVVDDQAVCDC
SRTGFRGKDCSQEIKFGLQCVLPVLLHDNDQGKYCCINTAKPLTEKDNN
VEGLAHLMMGDQGKSKGKEEYIATFKGSEYFCYDLSQNPIQSSSDEITL
SFKTLQRNGLMLHTGKSADYVNLALKNGAVSLVINLGSGAFEALVEPVN
GKFNDNAWHDVKVTRNLRQHSGIGHAMVNKLHCSVTISVDGILTTTGYT
QEDYTMLGSDDFFYVGGSPSTADLPGSPVSNNFMGCLKEVVYKNNDVRL
ELSRLAKQGDPKMKIHGVVAFKCENVATLDPITFETPESFISLPKWNAK
KTGSISFDFRTTEPNGLILFSHGKPRHQKDAKHPQMIKVDFFAIEMLDG
HLYLLLDMGSGTIKIKALLKKVNDGEWYHVDFQRDGRSGTISVNTLRTP
YTAPGESEILDLDDELYLGGLPENKAGLVFPTEVWTALLNYGYVGCIRD
LFIDGQSKDIRQMAEVQSTAGVKPSCSKETAKPCLSNPCKNNGMCRDGW
NRYVCDCSGTGYLGRSCEREATVLSYDGSMFMKIQLPVVMHTEAEDVSL
RFRSQRAYGILMATTSRDSADTLRLELDAGRVKLTVNLDCIRINCNSSK
GPETLFAGYNLNDNEWHTVRVVRRGKSLKLTVDDQQAMTGQMAGDHTRL
EFHNIETGIITERRYLSSVPSNFIGHLQSLTFNGMAYIDLCKNGDIDYC
ELNARFGFRNIIADPVTFKTKSSYVALATLQAYTSMHLFFQFKTTSLDG
LILYNSGDGNDFIVVELVKGYLHYVFDLGNGANLIKGSSNKPLNDNQWH
NVMISRDTSNLHTVKIDTKITTQITAGARNLDLKSDLYIGGVAKETYKS
LPKLVHAKEGFQGCLASVDLNGRLPDLISDALFCNGQIERGCEGPSTTC
QEDSCSNQGVCLQQWDGFSCDCSMTSFSGPLCNDPGITYIFSKGGGQIT
YKWPPNDRPSTRADRLAIGFSTVQKEAVLVRVDSSSGLGDYLELHIHQG
KIGVKFNVGTDDIAIEESNAIINDGKYHVVRFTRSGGNATLQVDSWPVI
ERYPAGNNDNERLAIARQRIPYRLGRVVDEWLLDKGRQLTIFNSQATII
IGGKEQGQPFQGQLSGLYYNGLKVLNMAAENDANIAIVGNVRLVGEVPS
SMTTESTATAMQSEMSTSIMETTTTLATSTARRGKPPTKEPISQTTDDI
LVASAECPSDDEDIDPCEPSSGGLANPTRAGGREPYPGSAEVIRESSST
TGMVVGIVAAAALCILILLYAMYKYRNRDEGSYHVDESRNYISNSAQSN
GAVVKEKQPSSAKSSNKNKKNKDKEYYV.

The terms “peptide” and “polypeptide” as used herein are intended to encompass any amino acid sequence including modified sequences. The terms “peptide” and “polypeptide” are specifically intended to cover naturally occurring proteins, as well as those, which are recombinantly or synthetically produced. The inhibitory peptides of the invention also include modified peptides (with amino acid substitutions, both conservative and non-conservative as described below) that have the same or improved activity as a wild-type or unmodified peptide. “Salts” of the peptides of the invention contemplated by the invention are physiologically and pharmaceutically acceptable organic and inorganic salts. The invention also provides conservative amino acid variants of the peptides according to the invention. Variants according to the invention also may be made that conserve the overall molecular structure of the encoded peptides. Given the properties of the individual amino acids comprising the disclosed peptide products, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved. The amino acids used in this invention are those, which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. These substitutions may enhance oral bioavailability, penetration into the islets, targeting to specific beta cell populations, immunogenicity, and the like. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1(—Alanine (A), Serine (S), Threonine (T);
2—Aspartic acid (D), Glutamic acid (E);
3—Asparagine (N), Glutamine (Q);
4—Arginine (R), Lysine (K);
5—Isoleucine (I), Leucine (L), Methionine (M), Valine (V); and
6—Phenylalanine (F), Tyrosine (Y), Tryptophan (W).

The peptides of the present invention may be produced by any method known in the art, including recombinant and synthetic methods. Synthetic methods include exclusive solid phase synthesis, partial solid phase synthesis, fragment condensation, or classical solution synthesis. Solid phase peptide synthesis procedures are well known to one skilled in the art and described, for example by John Morrow Stewart and Janis Dillaha Young, Solid Phase Polypeptide Syntheses (2nd Ed., Pierce Chemical Company, 1984). In some embodiments, synthetic peptides are purified by preparative high-performance liquid chromatography (Creighton T. (1983) Proteins, structures and molecular principles. WH Freeman and Co. N.Y.) The peptide sequence may be confirmed by amino acid sequencing using methods known to one skilled in the art.

In some embodiments, recombinant protein techniques are used to generate the peptide of the present invention. In some embodiments, recombinant protein techniques are used for generation of relatively long polypeptides (typically longer than 18 amino acids) or nucleic acid sequences or viral or bacterial vectors for vaccine formulation. Recombinant techniques are described for example by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Isolated polynucleotide sequences comprising at least one sequence encoding a peptide, peptide analog, peptide homolog conjugate or fusion protein of a modulator peptide are also included in the scope of the present invention. According to some embodiments, the polynucleotide sequence encoding the peptide or peptide analog, peptide homolog, is translationally linked to another polynucleotide sequence such as an RNA or DNA molecule and is recombinantly expressed within target cells. According to other embodiments, the polynucleotide sequence is part of a recombinant viral or bacterial vector.

The term “analog” refers to a molecule, which has the amino acid sequence according to the invention except for one or more amino acid changes. Analogs according to the present invention may include peptidomimetics. “Peptidomimetic” refers to a peptide modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with another covalent bond. A peptidomimetic according to the present invention may optionally comprise at least one bond, which is an amide-replacement bond such as urea bond, carbamate bond, sulfonamide bond, hydrazine bond, or any other covalent bond. The design of appropriate “analogs” may be computer assisted. Analogs are included in the invention as long as they remain pharmaceutically acceptable.

In some embodiment, the polypeptide comprises one or more amino acid substitutions, additions, or deletions. In some embodiment, the polypeptide comprises one or more substitutions corresponding to a conservative variant of the amino acid.

The terms “similarity”, “homology” and “sequence similarity/homology” may be interchangeably used and refer hereinafter to the level of identities between two homologous sequences when they are compared by aligning them using an alignment tool. As used herein, the sequence similarity is with respect to any one of the amino acid sequences disclosed herein and denoted by any one of SEQ ID NO: 1, SEQ ID NO: 2 SEQ ID NO: 3 and/or SEQ ID NO: 6.

The level of similarity or homology is determined by the number of identities in the amino acid sequence when they are aligned.

In some embodiments, the level of similarity between two amino acid sequences is the degree of identity between residues of the aligned sequence.

According to some embodiments, the amino acid sequence of the peptide modulator of the invention has at least 70%, 75%, 80%, 85%, 90%, 91%, 92%, 93%, 94%, 95%, 96%, 97%, 98%, 99%, or at least 99.9% sequence homology/identity/similarity to the amino acid sequence as set forth in any one of SEQ ID NO: 1, SEQ ID NO: 2 SEQ ID NO: 3 and/or SEQ ID NO: 6. Each possibility is a separate embodiment.

The term “plurality of isolated modulator peptides” refers to a combination/mixtures comprising one or more of the isolated modulator peptides of the invention as set forth in any one of SEQ ID NO: 1, SEQ ID NO: 2 SEQ ID NO: 3 and/or SEQ ID NO: 6 and/or homologs thereof including peptides having conservative substitutions and/or peptide analog/modified peptide thereof.

The terms “administration” and “administering” refer to providing or giving a subject a therapeutic agent (e.g. an isolated peptide or composition comprising the same), by any effective route. Exemplary routes of administration include, but are not limited to, injection or infusion (such as subcutaneous, intramuscular, intradermal, intraperitoneal, intrathecal, intravenous, intracerebroventricular, intrastriatal, intracranial and into the spinal cord), oral, intraductal, sublingual, rectal, transdermal, intranasal, vaginal and inhalation routes. Each possibility is a separate embodiment.

The terms “liver disorder”, “liver disease”, “liver-related disorder”, “liver-related condition” and “hepatic disease” are used interchangeably and refer to diseases and disorders that cause the liver to function improperly or to stop functioning. In some embodiments, the liver related condition may be selected from: non-alcoholic fatty liver disease (NAFLD), non-alcoholic steatohepatitis (NASH), cirrhosis, hepatitis A, hepatitis B, hepatitis C, hepatitis D, hepatitis E, liver adenoma, insulin hypersensitivity, liver cancer, and the like, or any combination thereof. Each possibility is a separate embodiment.

The terms “subject” and “patient” are interchangeable and as used herein refer to any individual suffering from a liver disorder.

The term “treatment” as used herein refers to both therapeutic treatment and prophylactic or preventative measures. In some embodiments, those in need of treatment include those already having a disorder as well as those in which the disorder is to be prevented. As used herein, the terms “prevent”, “reduce”, “attenuate”, “ameliorate”, “inhibit” may be used interchangeably.

The terms “composition” and “pharmaceutical composition” are used interchangeably and as used herein refers to any composition comprising at least one modulator peptide of the invention, or a composition which comprises cells (such as, NK cell) which harbor/include/express the modulator peptides. In some embodiments, the composition can include a plurality of peptides. In some embodiments, the composition can include a plurality of such cells. In some embodiments, the plurality of peptides may be a plurality of the same peptide, or a combination of several peptides. For example, in some embodiments, the composition may include peptide 1, peptide 2 and/or peptide 3.

The term “pharmaceutically acceptable carrier” refers to any carrier conventional used in the production of pharmaceutical compositions. Remington's Pharmaceutical Sciences, by E. W. Martin, Mack Publishing Co., Easton, Pa., 15th Edition, 1975, describes compositions and formulations suitable for pharmaceutical delivery of the compositions disclosed herein.

The term “antibody” is used in the broadest sense and includes monoclonal antibodies (including full length or intact monoclonal antibodies), polyclonal antibodies, multivalent antibodies, multi-specific antibodies (e.g., bi-specific antibodies), and antibody fragments long enough to exhibit the desired biological activity.

According to some aspects, the present invention provides novel isolated peptides, compositions including the same and uses thereof for treating liver disorders by modulating, interfering with, inhibiting and/or preventing NLGn4-Nrx1β interaction, in particular, by preventing protein-protein interaction (“PPI”) thereof.

In some embodiments, the isolated peptides may be derived from NLGN4X. The peptides may include different amino acid sequences that can vary in length, and may be derived from the same binding site as that of β-neurexin (i.e., the ligand of the NLG4NX receptor). In some embodiments, the active binding site is in a domain containing amino acids 359-364 in NLGN4X protein. In some embodiments, epitopes involved in binding may include, E361, L363, H267, Y463 and E270. In some embodiments, the isolated peptides are derived from E361 site epitope.

According to some embodiments the modulator peptide is used in a method of treating, attenuating and/or preventing progression of a liver disorder, the method includes administering a therapeutically effective amount of modulator peptide (or a composition including the same), the peptide having an amino acid sequence as denoted by any one of: SEQ ID NO: 1, SEQ ID NO: 2, SEQ ID NO: 3 and/or SEQ ID NO: 6, wherein said modulator peptide is capable of interfering with, inhibiting and/or preventing neuroligin 4 (NLGn4X)-Neurexin 1β (Nrx1β) interaction.

According to some embodiments, the modulator peptide, may be used in combination with at least one additional therapeutic agent, such as, PD1 and PDL1 inhibitors. In some embodiments, the modulator peptide may be comprised in a single composition with the additional therapeutic agent.

According to some embodiments, the modulator peptide may be a synthetic peptide or a recombinant peptide.

According to some embodiments, there are provided isolated modulator peptides for affecting the NLGn4-Nrx1β interaction, the modulator peptides have an amino acid sequence as set forth in SEQ ID NO: 6: EQGEFLNY. In some embodiments, the isolated modulator peptide may consist or comprise of SEQ ID NO: 6.

According to some embodiments, the isolated modulator peptide sequence of NLGn4-Nrx1β PPI may have an amino acid sequence as denoted by SEQ ID NO: 1: MEQGEFLNYD. In some embodiments, the isolated peptide having SEQ ID NO: 1 is designated “peptide 1”. In some embodiments, peptide 1 may comprise or consist of SEQ ID NO: 1.

According to some embodiments, the isolated modulator peptide sequence may have an amino acid sequence as denoted by SEQ ID NO: 2: QILMEQGEFLNYDIM. In some embodiments, the isolated peptide having SEQ ID NO: 2 is designated “peptide 2”. In some embodiments, peptide 2 may comprise or consist of SEQ ID NO: 2.

According to some embodiments, the isolated modulator peptide sequence may have an amino acid sequence as denoted by SEQ ID NO: 3: DPQILMEQGEFLNYDIMLGV. In some embodiments, the isolated peptide having SEQ ID NO: 3 is designated “peptide 3”. In some embodiments, peptide 3 may comprise or consist of SEQ ID NO: 3.

According to some embodiments, the present invention provides methods and compositions for treating, attenuating, and/or preventing progression of a liver disorder by activating natural killer cells (NK cells) and/or deactivating hepatic stellate cells (HSCs) by utilizing the modulator peptide(s) of the invention.

According to some embodiments, the present invention provides methods and compositions for treating, attenuating, and/or preventing progression of cancer condition in a subject in need thereof. In some embodiments, the cancer is a solid tumor. In some embodiments, the cancer maybe selected from, but not limited to: colorectal cancer, breast cancer, prostate cancer, liver cancer, or the like.

According to some embodiments, stimulation of NK cells with a modulator peptide of the invention can elevate/increase/improve the activity of the NK cells.

According to some embodiments, stimulation of NK cells with increasing concentrations of a modulator peptide can elevate/increase/improve the activity of the NK cells in a dose-dependent manner.

According to some embodiments, stimulation of NK cells with increasing concentrations of a modulator peptide of the invention can increase the expression levels of CD107a in NK cells. In some embodiments, the increase is dose-dependent.

According to some embodiments, stimulation of NK cells with increasing concentrations of NLGn4-Nrx1βPPI modulator peptide increases the levels of CD107a, in an inverse dose-dependent pattern. In some embodiments, the inverse dose-dependent pattern is that the levels of CD107a decrease at higher peptide concentration.

According to some embodiments, stimulation of hepatic stellate cells (HSCs) with increasing concentrations of NLGn4-Nrx1β PPI modulator peptide deactivates the cells.

According to some embodiments, stimulation of hepatic stellate cells (HSCs) with increasing concentrations of NLGn4-Nrx1β PPI modulator peptide decreases the levels of αSMA (Alpha Smooth Muscle Actin).

According to some embodiments, the present invention provides methods and compositions for treating, attenuating, and/or preventing progression of a liver disorder by activating natural killer cells (NK cells) and synergistically deactivating hepatic stellate cells (HSCs) by utilizing the isolated modulator peptides of the invention.

According to some embodiments, the inhibitory peptide is capable of mediating deactivation of HSCs, in particular, more prominently in the presence of activated NK cells.

According to some embodiments, pre-activation of NK cells with the inhibitory peptide(s) can render the cells more functional in deactivating HSCs cells.

As exemplified herein, it was surprisingly found that, according to some embodiments, pre-activation of NK cells with the inhibitory peptides of the invention can synergistically deactivate HSCs, as indicated, for example, by profound reduction in αSMA levels.

According to some embodiments, the effect of pre-activation of NK cells deactivating HSCs is dose-dependent. In some embodiments the effect reaches a steep 50% reduction in αSMA expression level at the highest concentration of NLGn4-Nrx1β PPI inhibitory peptide.

Surprisingly, according to some embodiments, inhibitory peptide activated NK cells, can synergistically function to downregulate/deactivate HSCs. According to some embodiments, the synergistic downregulation/deactivation of HSCs includes intense reduction in αSMA levels of HSCs co-cultured with NK cells pre-stimulated with NLGn4-Nrx1βPPI inhibitory peptide, relative to HSCs in monoculture stimulated NLGn4-Nrx1β PPI inhibitory peptide or to co-culture of unstimulated NK and HSCs.

According to some embodiments, following co-culturing with pre-activated-NK cells, two populations of HSCs may be identified, said populations characterized according to the αSMA expression levels, being medium or high (relatively).

Unexpectedly, according to some embodiments, NLGn4-Nrx1β PPI inhibitory peptides promote deactivation of HSCs, much more profoundly when first utilized to pre-stimulate NK cells killing activity.

According to some embodiments, there is provided a method of promoting anti-fibrotic effects in the liver, the method including administering any isolated modulatory peptide of the invention, or compositions including the same, to a subject in need thereof.

According to some embodiments, the NLGn4-Nrx1β inhibitory peptides can advantageously promote anti-fibrotic effects, by modulating NLGN4X/β-neurexin axis through functionally reducing hepatic stellate cell-mediated phagocytosis and/or increasing NK cells adherence-mediated killing of hepatic stellate cells.

According to some embodiments, the inhibitory peptides may disrupt the NLGN4X/β-neurexin axis by affecting different aspects of the HSCs/NK intercellular interaction such as, but not limited to: the ratio between different subpopulations of adhered cells expressing the NK cell-specific marker CD56+ and the HSCs activation marker αSMA, as either double-positive CD56+/αSMA+ or single positive CD56+/αSMA−.

According to some embodiments the inhibitory peptide may promote reduction of a subpopulation expressing CD56+/αSMA+ double-positive markers, that represent a subpopulation of NK cells that adhere to active HSCs, as were evaluated following the NK killing. In some embodiments, the effect of the inhibitory peptide is dose dependent.

Advantageously, in some embodiments, NK cells activated (treated) by the inhibitory peptides may be less engulfed into HSCs, compared to untreated HSCs/NK cells.

According to some embodiments, advantageously, inhibitory peptide-activated NK cells may be more functional in killing HSCs, compared to untreated HSCs/NK cells.

According to some embodiments, administration of an inhibitory peptide may promote increase in a subpopulation of NK cells expressing CD56+/αSMA− single-positive markers that represents a NK cell subpopulation that adheres to inactive HSCs cells. In some embodiments, the effect of the peptide is dose dependent.

According to some embodiments, there is provided a method of activating NK cells to thereby increase their affinity/binding/attachment/adherence to HSCs, the method including providing (administering) the cells with any of the modulatory peptides of the invention, or compositions including the same.

Advantageously, in some embodiments, inhibitory peptide-activated NK cells are more capable of attaching to inactive HSCs, relative to untreated HSCs/NK cells.

Advantageously, in some embodiments, inhibitory peptide-activated NK cells are more capable of attaching to active HSCs, to thereby deactivate HSCs, relative to untreated HSCs/NK cells interaction.

Advantageously, in some embodiments, inactive HSCs cells are less functional in engulfing and killing active-NK cells relative to inactive NK cells.

According to some embodiments, the inhibitory peptide of the invention can advantageously promote reduction in subpopulation of cells expressing CD56+/αSMA+ double-positive markers and increase in subpopulation expressing CD56+/αSMA− single-positive markers, relative to untreated HSCs/NK cells. In some embodiments, the effect is dose dependent.

According to some embodiments, there is provide a method for enhancing killing/inactivation of HSC cells and/or enhancing antifibrotic effects, the method includes activating NK cells with the modulatory peptides of the invention, or a composition including the same. In some embodiments, the activation is performed in-vivo. In some embodiments, the activation may be performed in-vitro and the activated NK cells may be administered (systemically and/or locally) to the subject.

According to some embodiments, inhibitory peptides-activated NK cells exhibit improved adherence to HSCs, thereby potentiating enhanced anti-fibrotic effects and/or killing of HSCs.

In some embodiments, inhibitory peptide-activated NK cells can cause HSCs to perform less phagocytosis of NK cells. In some embodiments, inhibitory peptide-activated NK cells may cause HSCs to be less functional (reduced functionality) in killing NK cells.

According to some embodiments, the inhibitory peptide can modulate/disrupt/block the NLGn4X/β-neurexin axis, by reducing, inhibiting, or preventing immune synapse between HSCs and NK cells that attenuates NK cell function.

According to some embodiments, there is provided a method of inhibiting or ameliorating liver fibrosis in a subject in the need thereof, the method includes administrating an inhibitory peptide of the invention, or a composition including the same to a subject in need thereof. According to some embodiments, the inhibitory peptide exhibit reduced to minimal side effects, such as, for example, liver toxicity.

According to some embodiments, the modulator peptide of the invention can inhibit liver fibrosis in vivo.

According to some embodiments, the modulator peptide does not affect appetite, liver toxicity and/or body weight. In accordance, in some embodiments a modulator peptide has no adverse effects and does not negatively affect the well-being of a treated subject, estimated by weight, food intake, water intake and liver weight.

According to some embodiments, the modulator peptide can be used to ameliorate several pathological characteristics evident through examination of the sera (serum) and liver biopsies of fibrotic liver. In some embodiments, the pathological characteristics may be selected from, but not limited to: serum levels of ALT, fibrotic liver inflammation, swelling levels of centrilobular hepatocytes, necrotic areas of high infiltrating inflammatory cells with steatosis, micro- and macrovascular steatosis, collagen deposition in perisinusoidal areas and/or the fibrous dense tissue, accumulation of thick fibrotic tissue, mRNA and/or protein levels of fibrotic biomarkers expressed in the liver, including αSMA, collagen I, CREBP, MMP-9, and the like, or any combination thereof.

In some embodiments, the modulator peptide can reduce levels of serum alanine transaminase (ALT).

According to some embodiments, the modulator peptide can ameliorate and/or prevent fibrotic liver inflammation.

According to some embodiments, the modulator peptide can prevent and/or reduce swelling of centrilobular hepatocytes and reduce necrotic areas of high infiltrating inflammatory cells with steatosis in fibrotic livers.

According to some embodiments, the modulator peptide can reverse histological alterations found in fibrotic livers.

According to some embodiments, the modulator peptide can prevent and/or reduce micro- and macrovascular steatosis in fibrotic livers.

According to some embodiments, the modulator peptide can prevent and/or reduce collagen deposition in perisinusoidal areas and/or the fibrous dense tissue in fibrotic livers.

According to some embodiments, the modulator peptide can prevent and/or reduce accumulation of thick fibrotic tissue in fibrotic livers.

According to some embodiments, modulator peptide may prevent and/or reduce mRNA and/or protein levels of fibrotic biomarkers expressed in the liver, such as, but not limited to αSMA, collagen I, CREBP, and MMP-9.

According to some embodiments the modulator peptide may modulate β-neurexin expression and/or activity in HSCs and/or F-actin expression of liver NK cells.

According to some embodiments, the modulator peptide can prevent or reduce β-neurexin expression and/or activity in fibrotic livers.

According to some embodiments, the modulator peptide can increase F-actin expression in fibrotic livers.

According to some embodiments, the modulator peptide can increase F-actin expression in fibrotic livers.

According to some embodiments, the modulator peptide can deactivate HSCs through inhibition of β-neurexin expression or activity.

According to some embodiments, the modulator peptide can increase F-actin expression in NK cells as evident by its co-localization with Natural Cytotoxicity Receptors (NKP46) inside NK cells.

According to some embodiments, the modulator peptide can restore NK activity through increased expression of F-actin, which is necessary for normal cellular function and motility.

According to some embodiments, there is provided a nucleic acid encoding for the isolated peptides of the invention. In some embodiments, there is provide a DNA construct/vector (such as, an expression vector) harboring or comprising a nucleic acid encoding for the peptides (optionally in addition to one or more regulatory sequences, non-coding sequences, and the like). In some embodiments, various suitable vectors are known to those skilled in art, and the choice of which depends on the function desired. Such vectors include, for example, plasmids, cosmids, viruses, bacteriophages and other vectors. In some embodiments, the polynucleotides and/or vectors harboring the same can be reconstituted into vehicles, such as, for example, liposomes for delivery to target cells. Any cloning vector and/or expression vector known in the art may be used, depending on the purpose, the host cell, and the like. Such vectors may be used for in-vitro and/or in-vivo introduction/expression.

According to some embodiments, the encoding nucleic acid molecules and/or the vectors disclosed herein may be designed for direct introduction or for introduction via carrier, such as, liposomes, viral vectors (adenoviral, retroviral) into target cells, such as, for example, NK cells.

According to some embodiments, there is provided a host cell harboring or expressing the isolated peptide(s) of the invention. In some embodiments, the host cell may be administered with the isolated peptide(s). In some embodiments, the host cell may be transformed/transfected with a vector or with a nucleic acid encoding for the isolated peptide. In some embodiments, the host cell is NK cell.

Accordingly, in some embodiments, the modulator peptide, or a composition comprising the same may be used for treatment of liver disorders and/or related cancer therapy.

According to some embodiments, the liver disorder may be selected from: fibrosis, non-alcoholic fatty liver disease (NAFLD), non-alcoholic steatohepatitis (NASH), cirrhosis, hepatitis, viral hepatitis, liver adenoma, insulin hypersensitivity, liver cancer, hepatocellular carcinoma, cholangiocarcinoma, liver metastasis, and the like, or any combination thereof.

According to some embodiments, the liver disorder is fibrosis. According to some embodiments, the liver disorder is non-alcoholic fatty liver disease (NAFLD). According to some embodiments, the liver disorder is non-alcoholic steatohepatitis (NASH). According to some embodiments, the liver disorder is cirrhosis. According to some embodiments, the liver disorder is hepatitis. According to some embodiments, the liver disorder is liver adenoma. According to some embodiments, the liver disorder is insulin hypersensitivity. According to some embodiments, the liver disorder is liver cancer. According to some embodiments, the liver disorder is hepatocellular carcinoma. According to some embodiments, the liver disorder is a combination of diseases.

According to some embodiments, any suitable route of administration to a subject may be used for the isolated peptides or the compositions of the present invention, including but not limited to, local and systemic routes. Exemplary suitable routes of administration include, but are not limited to: orally, intra-nasally, parenterally, intravenously, topically, enema or by inhalation. According to another embodiment, systemic administration of the composition is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention.

According to some embodiments, the administration may include any suitable administration regime, depending, inter alia, on the medical condition, patient characteristics, administration route, and the like. In some embodiments, administration may include administration twice daily, every day, every other day, every third day, every fourth day, every fifth day, once a week, once every second week, once every third week, once every month, and the like.

According to some embodiments, there are provided kits comprising the isolated peptides or composition comprising the same, as disclosed herein. In some embodiments, such kits/compositions can be used, for example, in the treatment of various liver-related conditions, such fibrosis, non-alcoholic fatty liver disease (NAFLD), non-alcoholic steatohepatitis (NASH), cirrhosis, hepatitis, viral hepatitis, liver adenoma, insulin hypersensitivity, liver cancer, hepatocellular carcinoma, cholangiocarcinoma, liver metastasis. In some embodiments, the kit may further include one or more reagents, buffers and/or instructions for using the same. In some embodiments, the composition may further include a pharmaceutically acceptable carrier.

According to some embodiments, the present invention provides methods and compositions for treating, attenuating, and/or preventing progression of a liver cancer by inhibiting proliferation and/or attenuating oncogenicity and/or increasing programmed cell death of liver cancer cells by utilizing the isolated modulator peptides of the invention.

According to some embodiments, there is provided a method of promoting anti-cancerous effects in the liver, the method including administering any isolated modulatory peptide of the invention, or compositions including the same, to a subject in need thereof, wherein the anti-cancerous effect comprises inhibiting proliferation and/or attenuating oncogenicity and/or increasing programmed cell death and/or reducing necrosis of liver cancer cells.

According to some embodiments, the modulator peptide of the invention inhibits/attenuates proliferation and/or oncogenicity of liver cancer cells.

According to some embodiments, the modulator peptide of the invention inhibits cell division of cancer cells by attenuating entry of the cancer cells into a state of S-phase or G2-M-phase.

According to some embodiments, the modulator peptide of the invention shifts cancer cells from existing at a state of S-phase or G2-M phase towards existing at a state of G1 phase.

According to some embodiments, the modulator peptide of the invention inhibits cell division of liver cancer cells (HCC) by attenuating entry of the cancer cells into a state of S-phase or G2-M-phase.

According to some embodiments, the modulator peptide of the invention shifts liver cancer cells (HCC) from existing at a state of S-phase or G2-M phase towards existing at a state of G1 phase.

According to some embodiments, the inhibiting/attenuating proliferation and/or oncogenicity of liver cancer cells using the modulator peptide of the invention is associated with attenuating entry of the cancer cells into a state of S-phase or G2-M-phase and increasing entry of the cancer cells into existing in a state of G1 phase.

According to some embodiments, the inhibiting/attenuating proliferation and/or oncogenicity of liver cancer cells using the modulator peptide of the invention is associated with reduces activation of Akt-signaling pathway and/or mTOR-signaling pathway.

According to some embodiments, the modulator peptide of the invention reduces activation of Akt-signaling pathway and/or mTOR-signaling pathway in cancer cells, thereby inhibiting proliferation and/or oncogenicity.

According to some embodiments, the modulator peptide of the invention reduces activation of Akt-signaling pathway and/or mTOR-signaling pathway in liver cancer cells (HCC), thereby inhibiting proliferation and/or oncogenicity.

According to some embodiments, the modulator peptide of the invention reduces the level of phosphorylated AKT/mTOR/P70S6K

According to some embodiments, the modulator peptide of the invention increases programmed cell death of cancer cells.

According to some embodiments, the modulator peptide of the invention reduces necrosis of cancer cells.

According to some embodiments, the modulator peptide of the invention increases programmed cell death of liver cancer cells (HCC).

According to some embodiments, the modulator peptide of the invention reduces necrosis of liver cancer cells (HCC).

According to some embodiments, the inhibiting/attenuating proliferation and/or oncogenicity of liver cancer cells using the modulator peptide of the invention is associated with increased programmed cell death or reduced necrosis of liver cancer cells

As used herein, the term “about” when referring to a measurable value such as an amount, a temporal duration, and the like, is meant to encompass variations of ±20% or in some instances ±10%, or in some instances ±5%, or in some instances ±%, or in some instances ±0.1% from the specified value, as such variations are appropriate to perform the disclosed methods.

As used herein, the terms “prevent”, “reduce”, “attenuate”, “ameliorate”, “inhibit” are used interchangeably.

As used herein, the terms “enhanced”, “increased”, “elevated” are used interchangeably.

As used herein, the singular forms “a,” “an” and “the” include plural referents unless the context clearly dictates otherwise.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Materials and Methods:

### Cell Culture Experiments:

Inhibitory Peptide synthesis: inhibitory peptide 1: MEQGEFLNYD (SEQ ID NO: 1), inhibitory peptide 2: QILMEQGEFLNYDIM (SEQ ID NO: 2), inhibitory peptide 3: DPQILMEQGEFLNYDIMLGV (SEQ ID NO: 3), and a control peptide having a scrambled sequence: LEGEDQNFYM (SEQ ID NO: 7) were synthesized by Pepscan Ltd. at a purity of 98.3%.

Cells: primary NK cells were isolated from patients having advanced fibrosis, human hepatic stellate cells (LX2) were commercially purchased.

Cell viability assay: In experiments involving NK cells, cell viability was assessed. The mean viability of cells, as measured by propidium iodide exclusion, was 92.7±1.5%.

Monoculture conditions: peptides were supplemented to media of cultured NK cells for 6 h stimulation.

Co-culture conditions: co-cultures conditions—peptides were supplemented to media of cultured NK cells for 6 h stimulation followed by a wash, trypsinization, and coculturing on LX2 cells for an additional 24 h.

Fluorescence-activated cell sorting (FACS): Staining colors of antibodies was as follows: αSMA>PE, CD107>FITC, CD56>. After analysis using LSR Fortessa analyzer, raw data was transferred to analysis program FCS Express v7.0.

Gene expression analysis by Flow cytometry: detection of proliferative or oncogenic markers expressed in Hep3B HCC cells, was performed according to a standard protocol, using antibodies for α-feto-protein (α-FP), Carboxy Fluorescein Succinimidyl Ester (CSFE), PDGFRA and MKI67 and of phosphorylated AKT/mTOR/P70S6K.

Cell cycle analysis by Flow cytometry: evaluation of cell cycle stage of Hep3B HCC cells, was performed according to a standard protocol, using Propidium-Iodide (PI)-staining of nuclei.

Cell death assessment by flow cytometry: evaluation of cell viability, necrosis and apoptosis of Hep3B HCC cells was performed according to a standard protocol, using Propidium-Iodide (PI)-staining of nuclei, phosphatidylserine staining using annexin V-conjugated to FITC, and CDKN2A.

Western Blot analysis: total protein extraction from of Hep3B HCC cells and WB analysis for β-neurexin were performed according to a standard protocol, using anti-human-β-neurexin and anti-GAPDH primary antibodies (R&D Systems) diluted 1:1000 and incubated overnight at 4° C.

### In-Vivo Experiments:

Animal model: 40 WT naïve C57BL/6J male mice (12 weeks) were either induced for acute hepatic fibrosis by i.p injections of 0.5 μl pure carbon tetrachloride CCl₄/g body weight (one to nine dilution in mineral oil) (CCl₄; Sigma, C-5331) 3×/week for four weeks as an advanced model of hepatic fibrosis, or left untreated (induced n=32, control n=8 per experiment). NLGn4-Nrx1β PPI modulator peptide 1 was i.p injected to CCl₄ induced mice as preventive doses from the beginning of the model starting at 1st week of CCl₄ at a concentration of 5 and 10 mg/mice 3×/week for four weeks (5 mg/mice n=8, 10 mg/mice n=8). A scrambled peptide was also i.p injected to CCl₄ induced mice (5 mg/mice n=8, 10 mg/mice n=8). Food and water intake were recorded every week. Mice were sacrificed two days after the final CCl₄ injection. To this end, the animals were weighed and anesthetized with inhaled 5% isoflurane for 10 seconds before cervical dislocation.

Histological and biochemical assessment of mice livers: The posterior one-third of the liver was fixed in 4% formalin for 24 hours and then paraffin-embedded in an automated tissue processor. Sections (7 mm) were stained for H&E for evaluating steatosis, necro-inflammatory regions and apoptotic bodies, 0.1% Sirius red F3B in saturated picric acid (Abcam, ab150681) as well as Masson's trichrome stain for connective tissue (Abcam, ab150686). Mice whole blood samples were collected at the sacrificing day, centrifuged at 3500 rpm for 10 minutes at 4° C. Serum ALT concentrations were determined using ELISA.

RNA isolation, cDNA preparation and real-time PCR: Total cellular RNA was isolated from liver tissue with 2 ml TRI Reagent (Bio LAB; Cat #90102331) per cm3 of tissue. The samples were homogenized for 5 minutes at room temperature. Chloroform at a volume of 0.2 ml (Bio LAB; Cat #03080521) was added to each sample, incubated for 15 minutes at room temperature and centrifuged (1,400 rpm) for 15 minutes at 4° C. For RNA precipitation, the supernatant in each sample was transferred to a new micro-centrifuge tube, 0.5 ml of isopropanol (Bio LAB; Cat #16260521) was added and incubated for 10 minutes at 25° C., and the tubes were centrifuged (12,000 rpm) for 10 minutes at 4° C. The supernatants were removed, and 1 ml of 75% ethanol was added to the pellet and centrifuged (7,500 rpm) for 5 minutes. The pellets were air dried at room temperature for 15 minutes, 50 μl of DEPC was added, and the samples were heated for ten minutes at 55° C. Preparation of cDNA was performed with a High-Capacity cDNA Isolation Kit (R&D; Cat #1406197). Real time PCR was performed with TaqMan Fast Advanced Master Mix (Applied Biosystems; Cat #4371130) for quantification of □SMA, Collagen I and Cyclic Adenosine Monophosphate-Responsive Element Binding Protein (CREBP) gene expressions, which were normalized to the expression of the housekeeping gene GAPDH.

Western blot analysis: Liver protein extracts were homogenized with buffer (50 mmol/L Tris-HCl [pH 7.61, 0.25% Triton-X 100, 0.15 M NaCl, 10 mM CaCl₂) and complete mini EDTA-free protease inhibitor cocktail (Roche Diagnostics, Mannheim, Germany). Next, proteins (20 μg per lane) were resolved on a 10% (w/v) SDS-polyacrylamide gel (Novex) under reducing conditions. For immunoblotting, proteins were transferred to a Protran membrane. Blots were then incubated for 1 hour at room temperature in a neutralizing buffer containing 5% skim milk (w/v). Next, the blots were incubated with mice anti-human/mice αSMA, rabbit anti-human-β-Nrxn, goat anti-mice F-actin and sheep anti-human/mice MMP-9. All primary antibodies (R&D Systems) were diluted 1:1000 overnight at 4° C. The blots were subsequently incubated with peroxidase-conjugated anti-mice, -rabbit, -goat, -rat, -sheep, or IgG (Abcam, diluted 1/5000) for 1.5 hour at room temperature. Immunoreactivity was detected using an ECL kit (Abcam).

Immunofluorescence staining: For deparaffinization, paraffin-embedded sections were placed at 60° C. for 15 minutes, incubated in xylene at room temperature for 15 minutes, and then transferred sequentially to 100% EtOH, 95% EtOH, 70% EtOH, and 50% EtOH for four minutes each at room temperature. Sections were rinsed in deionized water and stored in phosphate buffer saline (PBS). For antigen retrieval, a buffer (10 mM citrate, pH 6.2, 2 mM EDTA, and 0.05% Tween 20) was used. Mice livers were outlined with 100 μl of KASBLOCK liquid blocker to minimize the volume of antibody solution needed for staining. Samples were incubated overnight at 4° C. with goat anti-mice F-actin (diluted 1:50), rabbit anti-mouse NKP46 (diluted 1:100). Following washing with PBS, secondary antibodies conjugated with Cy-3, Cy-2, or Alexa-flour 647 were added for one hour at room temperature, and image capture was performed. Samples were viewed and imaged with a Zeiss LSM 710 confocal laser-scanning system (Zeiss) attached to a Zeiss Axiovert 135M microscope, equipped with a Plan-apochromat Zeiss 63×lens. An argon laser (488 nm excitation) was used to detect green fluorescence, and an Alexa Fluor laser (552 nm) to detect red fluorescence.

Fluorescence activated cell sorting (FACS): Mice liver NK cells were characterized as NK1.1. For assessment of NK activations, CD107a; LAMP1 (lysosomal-associated membrane protein-1) were used. An isotype IgG labeled with the relevant fluorochrome was used as a control. Stained cells were analyzed with a flow cytometer (BD LSR Fortessa™, Becton Dickinson, Immunofluorometry systems).

Statistical Analysis: Statistical differences were analyzed with two-tailed unpaired Student's either t-test (for comparison between two groups) or one-way analysis of variance (one-way ANOVA with Newman-Keuls post-tests among multiple groups) in GraphPad Prism 5.0 (GraphPad Software, La Jolla, CA). For the in vitro study validation, every experiment was repeated three times each with 4 replicates.

### Example 1—NLGn4-Nrx1β Inhibitory Peptides Stimulate the Activity of NK Cells and Reduce the Activity of LX2 Cells, in Monocultures

Three peptides potentially having an inhibitory effect on NLGn4-Nrx1β PPI were generated. The three peptides (peptide 1, peptide 2, and peptide 3) consist of different amino acid sequences (SEQ ID NO: 1, SEQ ID NO: 2, and SEQ ID NO: 3, respectively) that vary in length, all derived from the same binding site for the NLGn4X ligand β-neurexin. The active binding site includes a domain containing the amino acids 359-364 in NLGn4X and the epitopes critical for binding are E361, L363, H267, Y463, and E270. Hence, the peptides were screened for their potential inhibitory effect on NLGn4-Nrx1β PPI, first, by measuring the activity of human natural killer (NK) cells and human hepatic stellate cells (LX2), in monocultures.

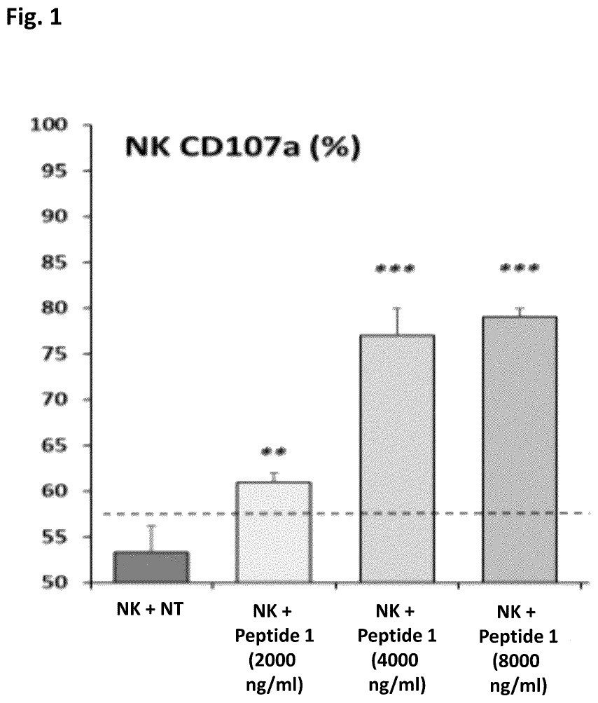

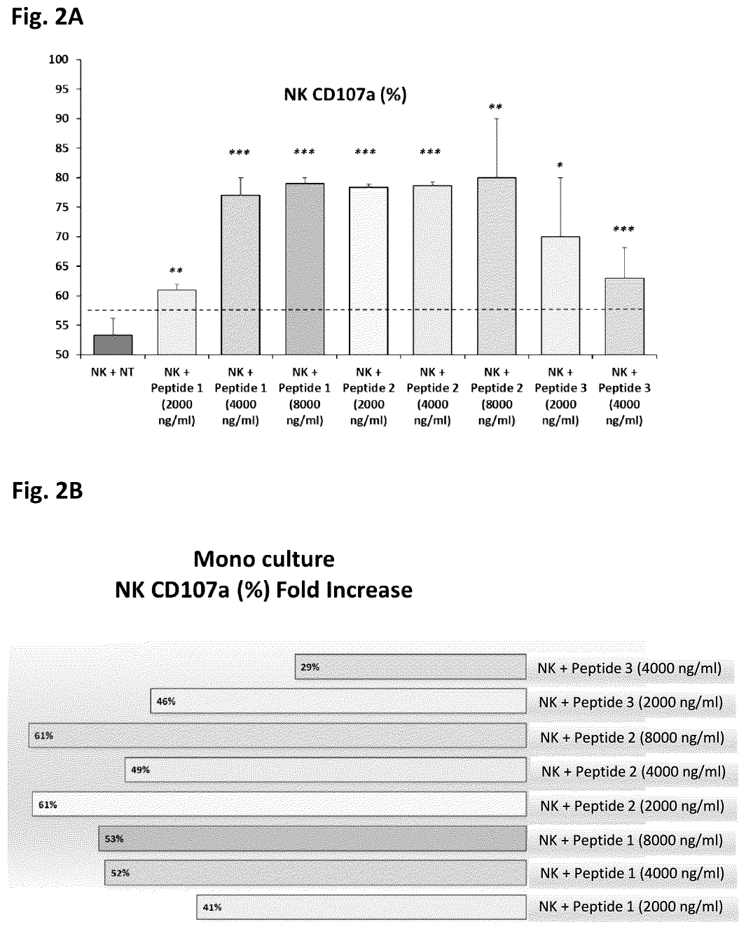

Initially, peptides were screened for their effect on the activity of NK cells, which was evaluated by measuring the levels of CD107a activation marker. Monocultures of NK cells were stimulated with increasing concentrations of the potentially inhibitory NLGn4-Nrx1β PPI peptides (FIG. 1 and FIGS. 2A-B). Stimulation of NK cells with increasing concentrations of NLGn4-Nrx1β PPI Peptide 1 elevated the activity of the cells in a dose-dependent manner, as indicated by the increase in levels of CD107a, shown in FIG. 1. Similar to the effect observed with peptide 1, stimulation of NK cells with NLGn4-Nrx1β PPI Peptide 2 and NLGn4-Nrx1β PPI peptide 3 with the same increasing concentrations also elevated the activity of the cells, as indicated by the increase in the levels of CD107a, shown in FIG. 2A and FIG. 2B. However, the effect of peptide 2 on NK cells activity was not dose-dependent. Notably, with peptide 3 the increase in activity followed an inverse dose-dependent pattern, according to which the levels of CD107a decreased at higher peptide concentrations, indicating peptide 3 may be toxic to the cells.

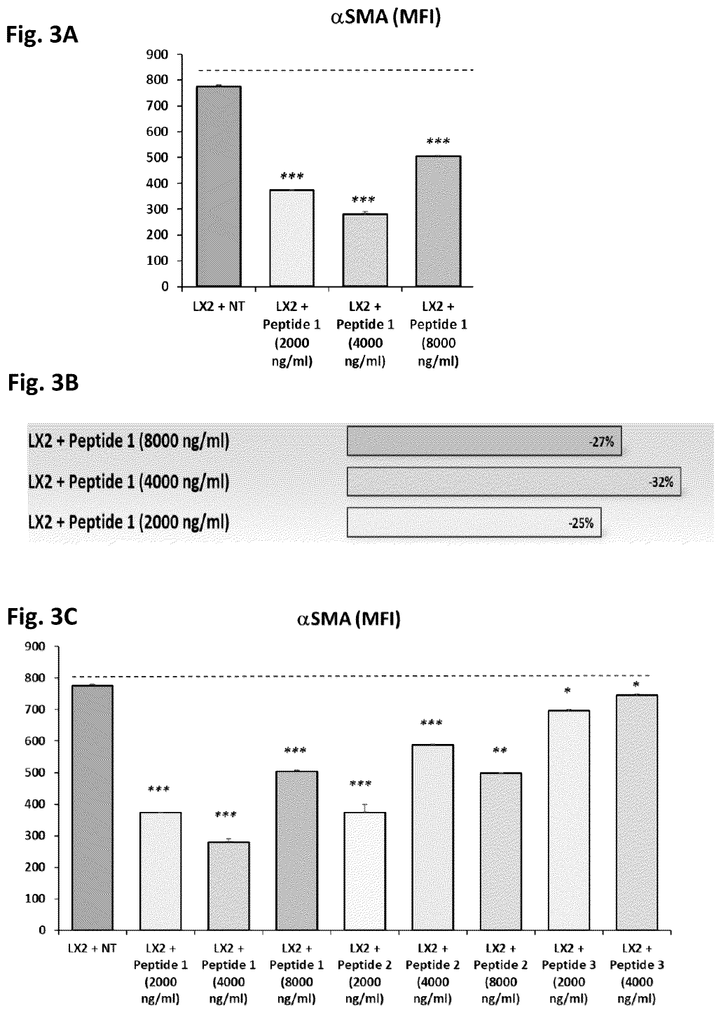

Following that, the NLGn4-Nrx1β PPI peptides were screened for their effect on the activity of LX2 cells, which was evaluated by measuring the levels of αSMA activation marker. Monocultures of LX2 cells were stimulated with increasing concentrations of the NLGn4-Nrx1βPPI peptides (FIGS. 3A-C). Stimulation of LX2 cells with increasing concentrations of peptide 1 deactivated the cells, as indicated by the decrease in levels of αSMA shown in FIG. 3A and FIG. 3B. Similar to the effect observed with peptide 1, stimulation of LX2 cells with peptide 2 and peptide 3 with the same increasing concentrations also resulted in deactivation of the cells, as indicated by the reduction in the levels of αSMA, shown in FIG. 3C.

### Example 2—NLGn4-Nrx1β PPI Inhibitory Peptides Synergistically Deactivate Co-Cultured LX2 Cells Through Activated NK Cells

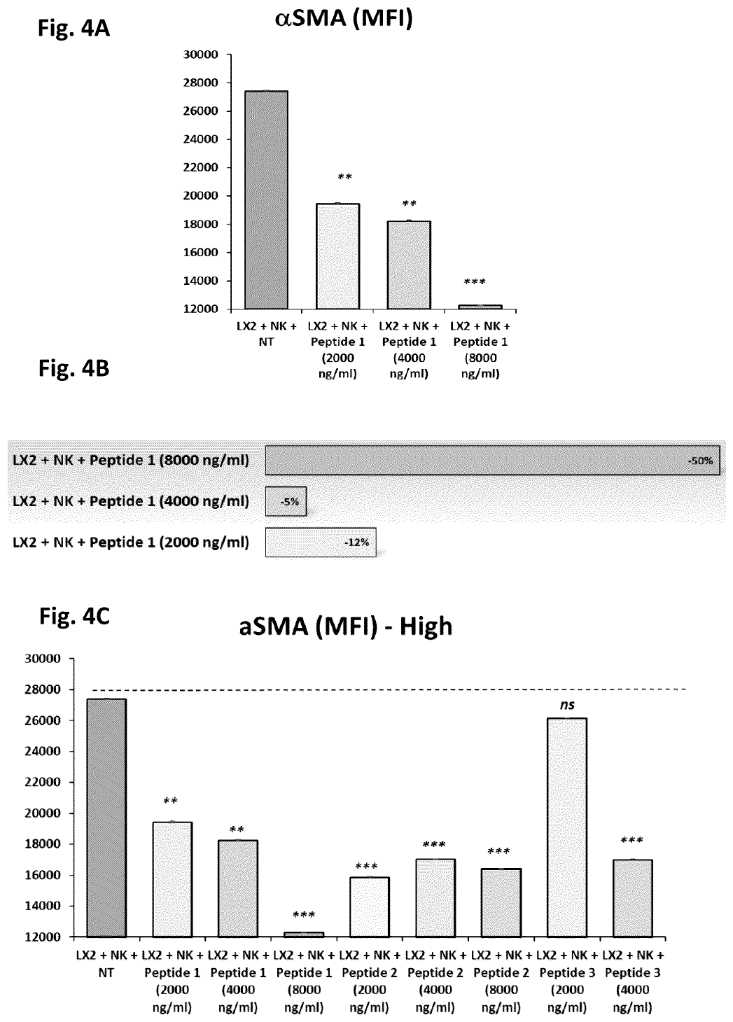

To test if NLGn4-Nrx1β PPI inhibitory peptides mediated deactivation of LX2 cells is more prominent in the presence of NK cells, a co-culture setting was established for LX2 cells in the presence or absence of activated NK cells (FIGS. 4A-C).

In this setting, NK cells were stimulated with increasing concentrations of NLGn4-Nrx1βPPI inhibitory peptides before co-culturing them with LX2 cells, to examine whether pre-activation makes them more functional in deactivating LX2 cells. Surprisingly, under co-culturing conditions, peptide 1 activated NK cells and synergistically deactivated LX2 activity, as indicated by the profound reduction measured in the levels of αSMA, shown in FIG. 4A.

Moreover, the effect was dose-dependent and reached a steep 50% reduction in αSMA expression level at the highest concentration of peptide 1 (FIG. 4B).
This intense further reduction in αSMA levels of LX2 cells co-cultured with peptide 1-stimulated NK cells, relative to peptide 1-stimulated LX2 cells in monoculture or to co-culture of unstimulated NK and LX2 cells, demonstrates a surprising and outstanding synergistic functionality for peptide 1—activated NK cells in downregulating/deactivating hepatic stellate cells.

Similar to the effect observed with peptide 1, co-culturing of LX2 cells with NK cells pre-activated with the same increasing concentrations of peptide 2 and peptide 3 also downregulated the activity of the LX2 cells, as indicated by reduction in the levels of αSMA, shown in FIG. 4C. However, the effect of peptide 2 on NK cells activity was not dose-dependent, while that of peptide 3 did follow a dose-dependent pattern.

Notably, FACS analysis indicated two populations of LX2 cells in respect to αSMA levels expression presenting either medium (data not shown) or high expression level, following co-culturing with pre-activated-NK cells.

### Example 3—NLGn4-Nrx1β PPI Inhibitory Peptides Modulate NLGN4X/β-Neurexin Axis by Functionally Reducing Hepatic Stellate Cell Activation and Increasing NK Cells Adherence-Mediated Killing of Hepatic Stellate Cells

As evident by the profound decrease in αSMA levels of expression in the LX2-NK co-culture setting, NK cells pre-activated by stimulation with NLGn4-Nrx1β PPI inhibitory peptides promote a strong deactivation of hepatic stellate cells.

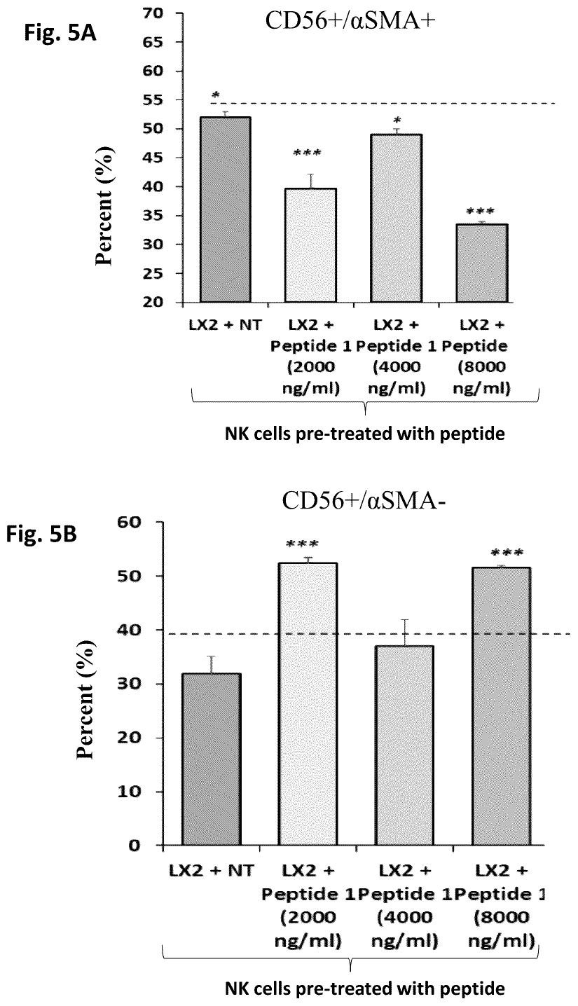

To further test the effect of NLGn4-Nrx1βPPI inhibitory peptides on the NLGN4X/β-neurexin axis different aspects of the LX2/NK intercellular interaction were investigated. The tendency of NLGn4-Nrx1β PPI inhibitory peptide-activated NK cells to physically interact with different populations of LX2 cells was assayed by FACS-sorting, testing only the adherent portion of cells in the co-culture of LX2/peptide-activated-NK cells (FIG. 5A-B). The effects of increasing concentrations of peptides on two populations of cells were assessed based on sorting of the NK cell-specific CD56+ marker and the hepatic stellate cell activation marker αSMA, as either double-positive CD56+/αSMA+ or single positive CD56+/αSMA−.

Results with increasing concentrations of peptide 1 show that, relative to untreated control of co-cultured LX2/NK cells, pre-activation of NK cells with NLGn4-NrxlP PPI inhibitory peptide 1 promotes a reduction in a subpopulation expressing CD56+/αSMA+ double-positive markers that represent a subpopulation of NK cells that adhere to active LX2 cells (FIG. 5A). This indicates that NLGn4-Nrx1βPPI inhibitory peptide-1-activated NK cells significantly reduced HSC activation and are less engulfed by the LX2 cells.

Results with increasing concentrations of peptide 1 show that, relative to untreated control of co-cultured LX2/NK cells, pre-activation of NK cells with NLGn4-NrxlP PPI inhibitory peptide 1 promotes an increase in a subpopulation expressing CD56+/αSMA− single-positive markers that represent a subpopulation of NK cells that adhere to unactive LX2 cells (FIG. 5B). This indicates that NLGn4-Nrx1β PPI inhibitory peptides 1-activated NK cells are more adhered to deactivating LX2 cells.

These results too indicate that NLGn4-Nrx1βinhibitory peptides-activated NK cells adhere more to LX2 cells thereby possibly potentiating more anti-fibrotic effects and killing of LX2 cells.

It is evident that NLGn4-Nrx1βPPI inhibitory peptides can modulate/disrupt/block NLGn4X/0-neurexin axis, by reducing, inhibiting, or preventing immune synapse between LX2 and NK cells that attenuates NK cell function.

### Example 4—NLGn4-Nrx1β PPI Peptides Inhibits Liver Fibrosis in In Vivo

Following the establishment of the CCl₄-induced acute hepatic fibrosis model in mice, two days after the final CCl₄ and NLGn4-Nrx1β PPI modulator peptide 1 or scrambled peptide (SEQ ID NO: 7) injections, mice livers were harvested, biopsies for histological staining and examination of liver pathology were taken, and extraction of mRNA and protein was performed for assessment of gene expression using RT-PCR, western blot and ELISA assays, as well as extraction of isolated NK cells for immunofluorescence staining. Genes whose expression was analyzed included F-actin in isolated NK cells, β-neurexin, and a panel of liver fibrosis markers (such as alpha-SMA expression—a biomarker of HSC activation, collagen produced by HSC, CREBP, and MMP-9). In addition, sera were collected for detection of inflammation and expression of alanine transaminase (ALT—a biomarker for liver health).

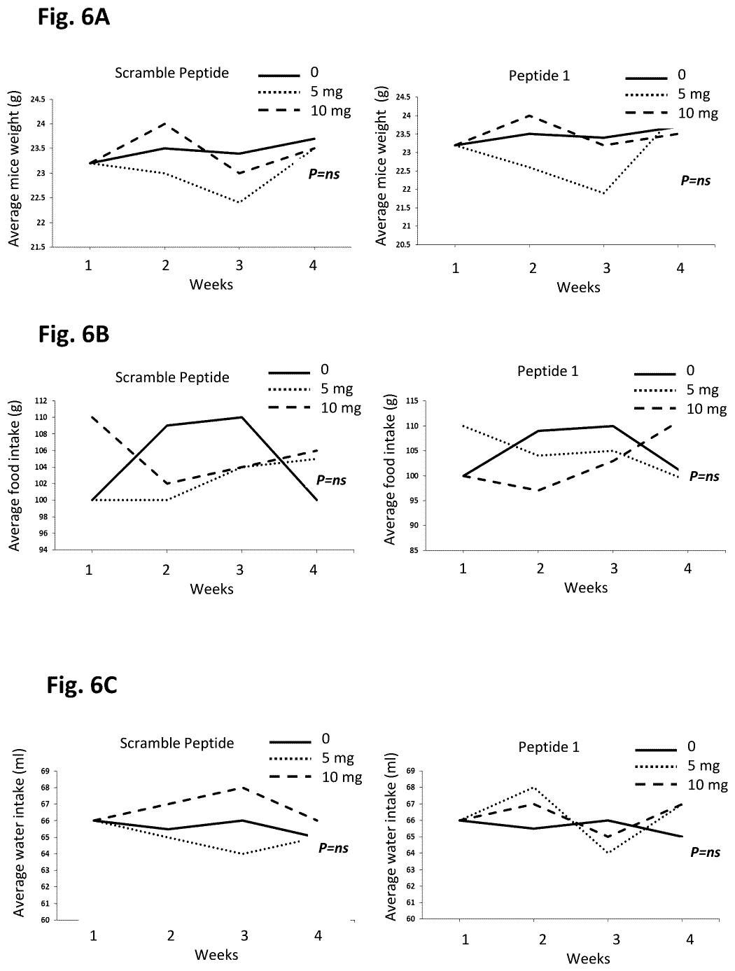

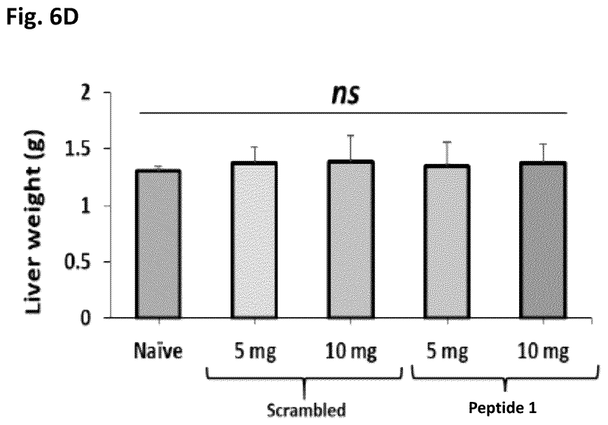

As seen in FIGS. 6A-6D, a follow up on the potential adverse effects of NLGn4-Nrx1β PPI modulator peptide 1 (appetite, liver toxicity, body weights) found the peptide safe for use, without significantly affecting mice well-being as estimated by average mice weight (FIG. 6A), food intake (FIG. 6B), water intake (FIG. 6C) and liver weight (FIG. 6D).

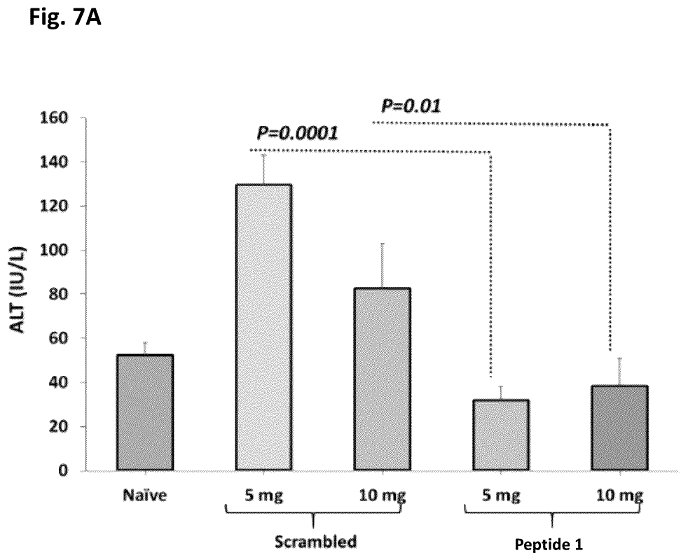

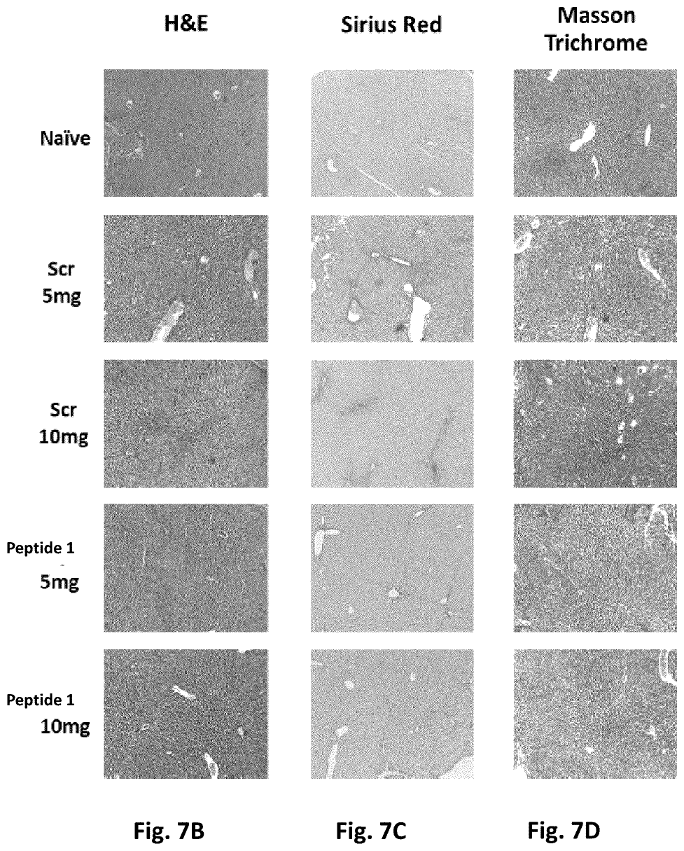

As seen in FIG. 7A-7D, NLGn4-Nrx1β PPI modulator peptide 1 ameliorated several pathological characteristics that were evident through the examination of the sera and liver biopsies of mice induced for acute liver fibrosis in the CCl₄ model.

Mice induced for acute liver fibrosis that were treated with NLGn4-Nrx1β PPI modulator peptide 1 had reduced levels of serum ALT, similar to the levels of the naïve untreated mice, compared to mice that received scramble control peptide and had a significant increase in ALT levels (FIG. 7A).

These results demonstrate that treatment with NLGn4-Nrx1βPPI modulator peptide 1 ameliorates and even prevented liver inflammation as evidenced by the reduction in serum ALT levels.

Mice induced for acute liver fibrosis that were treated with NLGn4-Nrx1β PPI modulator peptide 1 had reduced swelling of centrilobular hepatocytes and reduced necrotic areas of high infiltrating inflammatory cells with steatosis, compared to CCl₄ induced livers (FIG. 7B).

These results demonstrate that treatment with NLGn4-Nrx1βPPI modulator peptide 1 reversed histological findings present at CCl₄ induced fibrotic livers, with a significant reduction in micro- and macrovascular steatosis, as evidenced by H&E staining.

In addition, Sirius Red staining of liver biopsies indicates that mice induced for acute liver fibrosis and treated with scrambled peptide had increased collagen deposition in perisinusoidal areas, compared to naïve uninduced mice, while treatment with NLGn4-Nrx1β PPI modulator peptide 1 resulted in a remarkable reduction in the fibrous dense tissue of the stained area (FIG. 7C).

Moreover, Masson's trichrome staining of NLGn4-Nrx1β PPI modulator peptide 1 treated mice showed minimal accumulation of thick fibrotic tissue relative to CCl₄-induced livers treated with scrambled peptide, compared to CCl₄ untreated mice. (FIG. 7D).

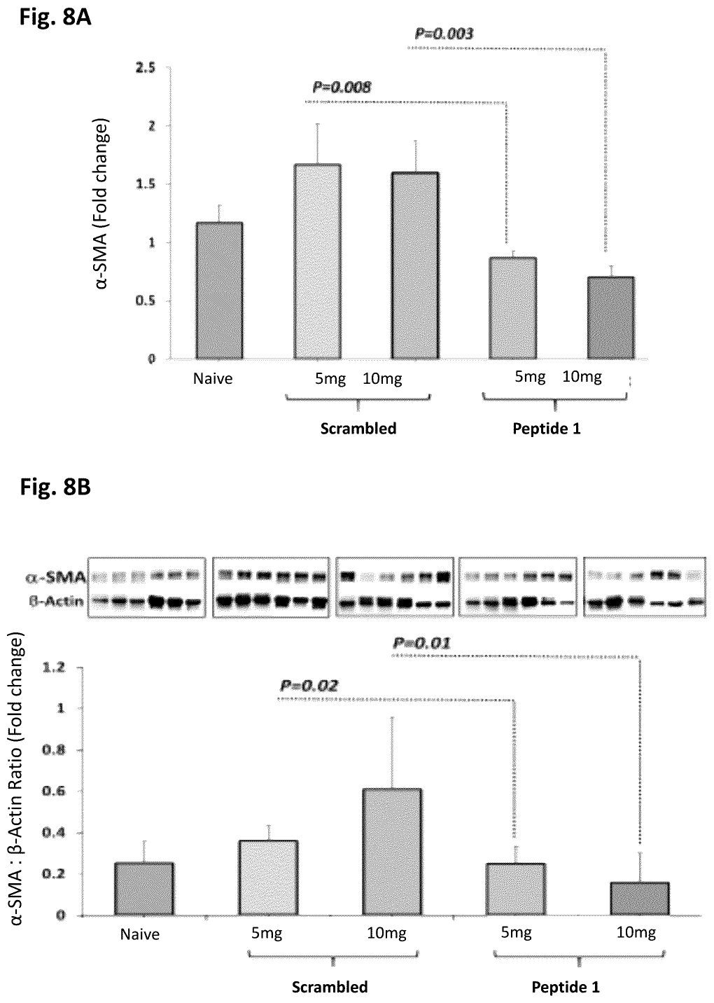

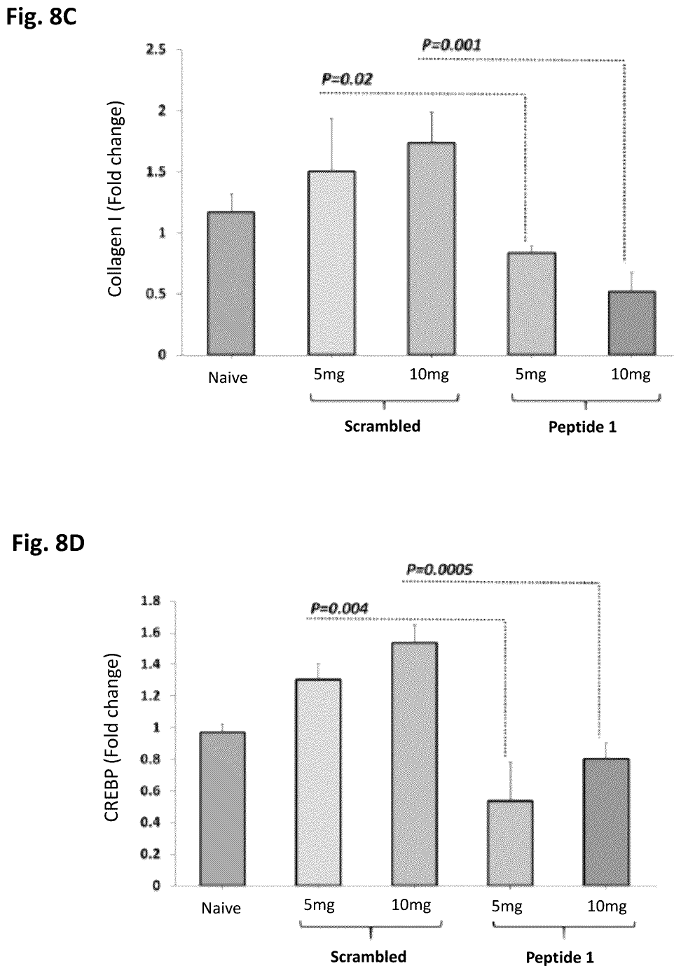

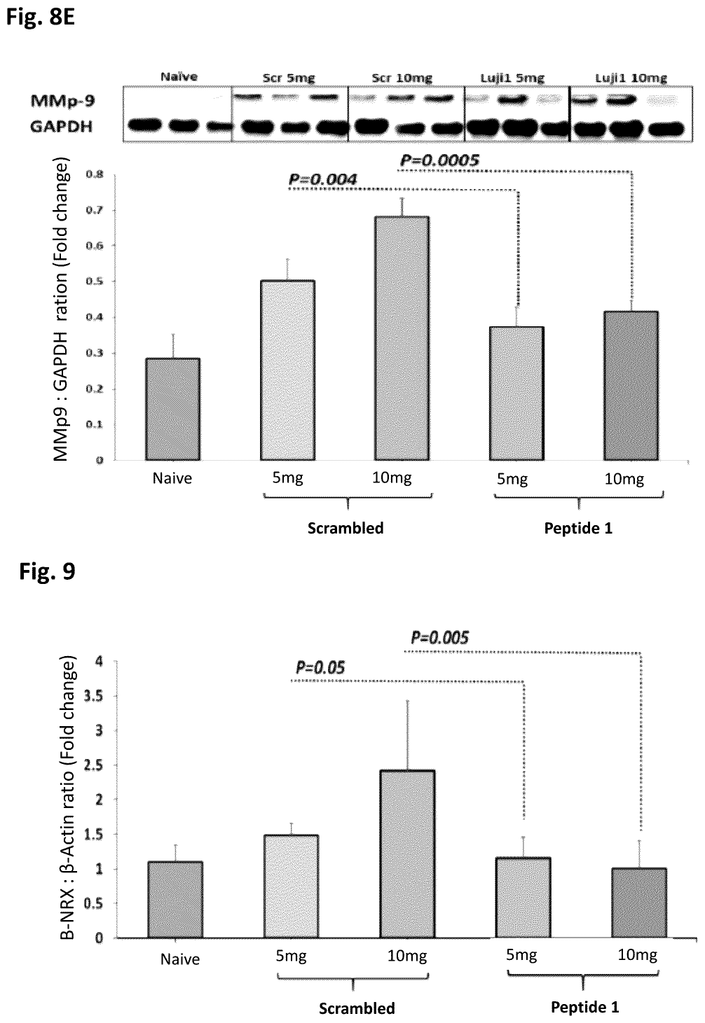

As seen in FIGS. 8A-8E, mice induced for acute liver fibrosis showed elevated mRNA and protein levels of a panel of fibrotic biomarkers expressed in the liver, whereas treatment with NLGn4-Nrx1β PPI modulator peptide 1 prevented this increase, as indicated by the relative reduction in the levels of αSMA mRNA (FIG. 8A) and protein (FIG. 8B), collagen I mRNA (FIG. 8C), CREBP mRNA (FIG. 8D), and MMp-9 protein (FIG. 8E).

This result showing a reduction in a panel of fibrotic biomarkers is in line with the herein above histological findings that treatment with NLGn4-Nrx1β PPI modulator peptide reversed histological findings present at CCl₄ induced fibrotic livers.

### Example 5—NLGn4-Nrx1β PPI Peptide In Vivo Modulates β-Neurexin Expression of HSCs and F-Actin Expression of Liver NK Cells

β-neurexin was shown to be expressed mainly in activated liver HSCs that display high levels of fibrotic markers. As seen in FIG. 9, mice induced for acute liver fibrosis showed elevated levels of β-neurexin, relative to naïve mice, when treated with scrambled peptide. NLGn4-Nrx1β PPI modulator peptide 1 prevented this increase.

In view of the abovementioned amelioration of liver fibrosis in these mice model and the observed reduction in the levels of the activation marker αSMA in response to treatment with NLGn4-Nrx1β PPI modulator peptide 1, this instant result may indicate that NLGn4-Nrx1βPPI modulator peptide 1 directly deactivates HSCs through inhibition of β-neurexin.

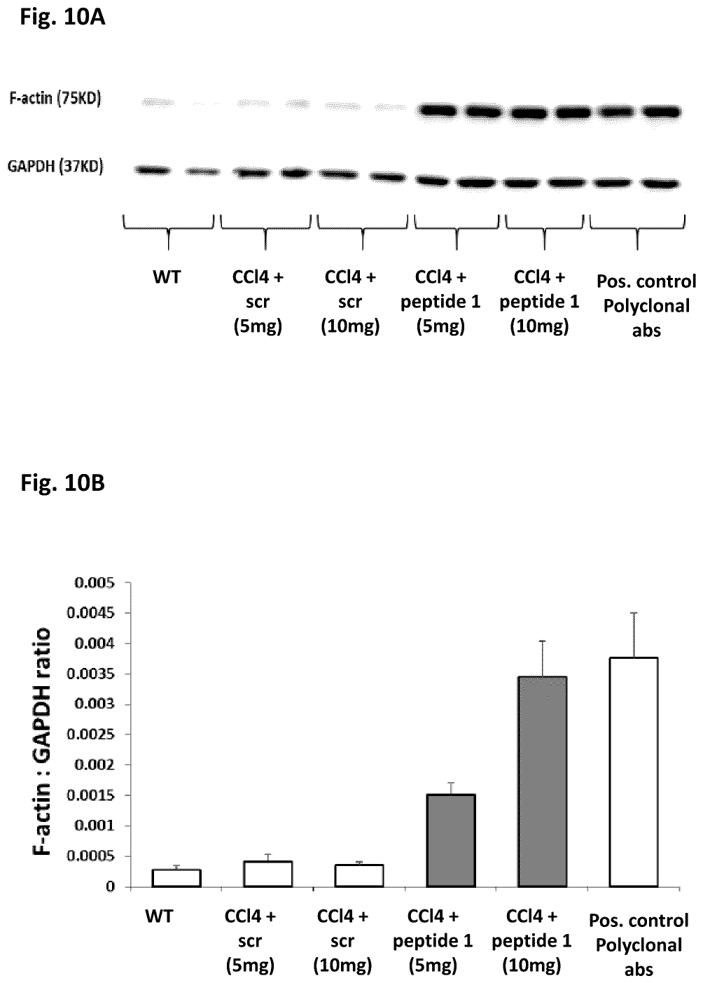

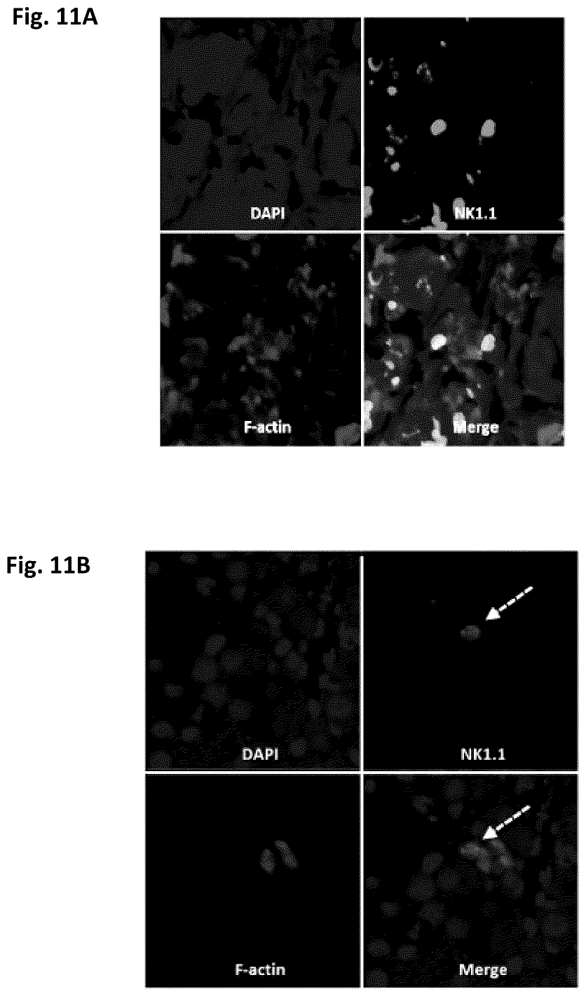

As seen in FIGS. 10A-10B and FIGS. 11A-11B, NLGn4-Nrx1β PPI modulator peptide 1 induced a strong upregulation in the expression of the cytoskeletal protein F-actin in NK cells of CCl₄ induced fibrotic livers, relative to naïve mice or mice with induced livers treated with scrambled peptide. This result was observed and quantified in isolated liver NK cells (FIG. 10A (Western Blot Analysis) and 10B (quantification Graph) (NK cells were isolated by FACS sorting based on CD107a). Further, visualization of immune-stained liver tissue show that wherein F-actin is seen at higher fluorescence intensities and co-localized with Natural Cytotoxicity Receptors (NKP46) inside NK cells (FIG. 11A) of CCl₄ induced mice treated NLGn4-Nrx1β PPI modulator peptide 1, it is not seen inside NK cells of CCl₄ induced mice treated with a scrambled peptide (FIG. 11B).

These results indicate that NLGn4-Nrx1po modulator peptide 1 restores NK activity through increased expression of F-actin, which is necessary for normal cellular function and motility, thereby, further strengthening the effectiveness of the modulator peptides for treatment of liver disorders.

### Example 6—NLGn4-Nrx1β PPI Peptide Modulates Proliferation of Hepatocellular Carcinoma (HCC) Cells In Vitro

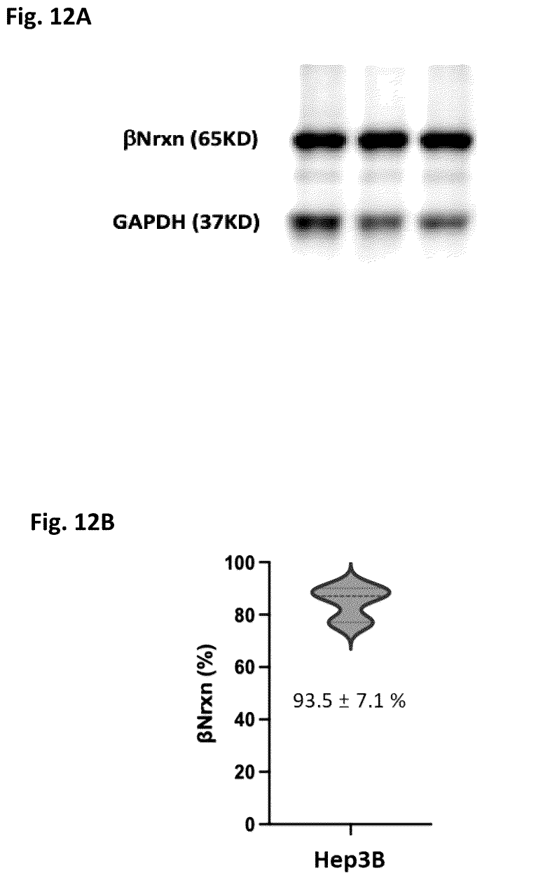

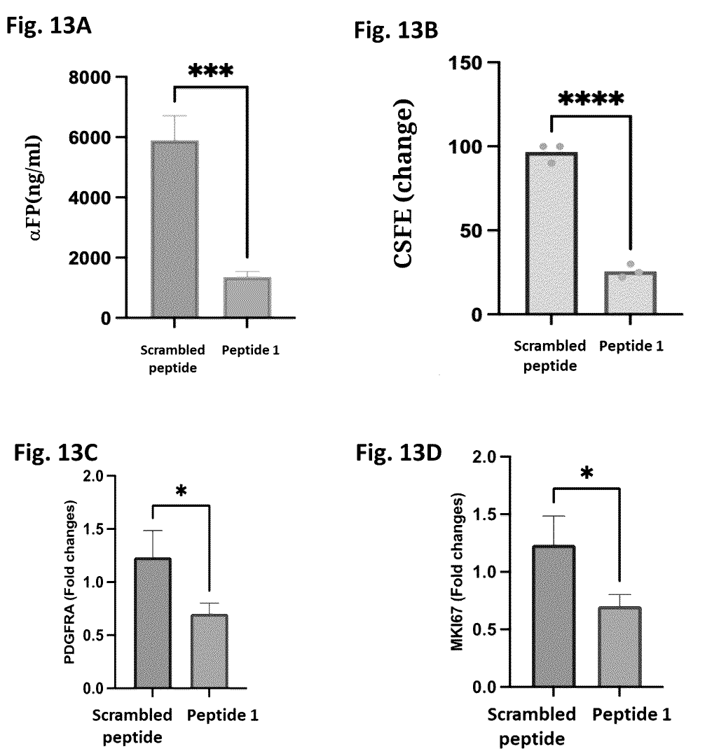

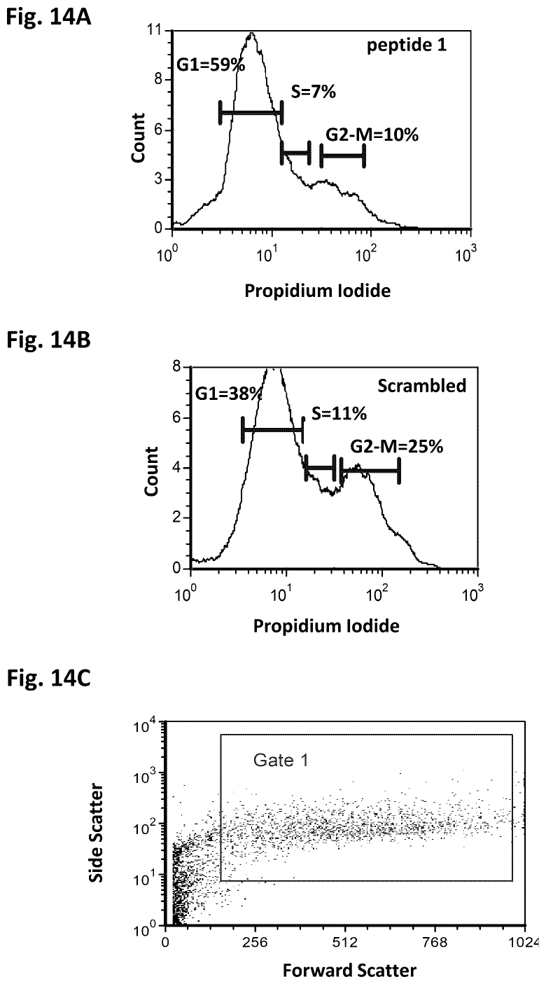

The effect modulation of NLGn4-Nrx1βPPI exerts on proliferation and carcinogenicity of liver cancer cells was evaluated in-vitro in the Hepatocellular carcinoma (HCC) cell line Hep3B, by exposing the cells to 8000 ng/ml of peptide 1 and performing gene expressions analyses of β-neurexin (FIG. 12A-12B) and of specific proliferation markers (FIG. 13A-13D) and analysis of the stage of cell cycle (FIG. 14A-14C).

First, β-neurexin expression was evaluated in Heb3B cells or normal hepatocytes treated with peptide 1. As indicated by western blot (FIG. 12A) and flow cytometry (FIG. 12B) analyses, Hep3B cells treated with peptide 1 (SEQ ID NO: 1) express transmembrane surface of β-neurexin at higher levels (approximately 95%) compared with normal hepatocytes that express β-neurexin at much lower levels (approximately 6.5%, not shown).

Next, expression of α-feto-protein (α-FP), a marker for carcinogenicity, and of Carboxy Fluoroscein Succinimidyl Ester (CSFE), PDGFRA and MKI67 markers for proliferation, was evaluated in Hep3B cells treated with peptide 1 or with a control peptide having a scrambled sequence (SEQ ID NO: 7). ELISA and flow cytometry analyses indicated a strong and significant reduction in the expression levels of all markers following treatment with peptide 1. Specifically, α-FP and CSFE showed about 3-fold to about 4-fold reduction in expression levels (FIGS. 13A-13B), and PDGFRA and MKI67 showed about 2-fold reduction in expression levels (FIGS. 13C-13D).

To ratify attenuation of the proliferative capabilities of HCC cells following modulation by peptide 1, the stage of the cell cycle of Hep3B cells treated with 8000 ng/ml peptide 1 or with a control peptide having a scrambled sequence was evaluate using flow cytometry and Propidium Iodide (PI)-staining. As seen in FIGS. 14A-14C cell cycle analysis of G1, S and G2-M phases are indicative of a 2.5-fold delay/decrease, from 25% to 10%, in the percentage of cells existing at a state of G2-M phase performing mitosis/dividing, or about 2-fold decrease, from 11% to 7%, in the percentage of cells existing at a state of S-phase performing replication/synthesis of DNA, while on the other hand the percentage of cells existing at a state of G1 is increased by about 2-fold, from 38% to 59%.

These results suggest that peptide 1 inhibits/attenuates proliferation/cell division of Hep3B HCC cells as estimated by decreased expression of proliferation markers CSFE, PDGFRA and MKI67 which was associated with a decreased expression of α-FP which is indicative of a reduction in oncogenicity.

Advantageously, these results suggest that peptide 1 inhibits/attenuates/alters cell division/proliferation of Hep3B HCC cells by affecting/modulating cellular processes downstream of β-neurexin, including a notable and unexpected effect on the cell cycle including reduced cell division, manifested as a cellular shift from existing at a state of S-phase or G2-M phase towards existing at a state of G1 phase.

### Example 7—NLGn4-Nrx1β PPI Peptide Modulates Cellular Proliferation and/or Oncogenic Signaling of Hepatocellular Carcinoma (HCC) Cells In Vitro

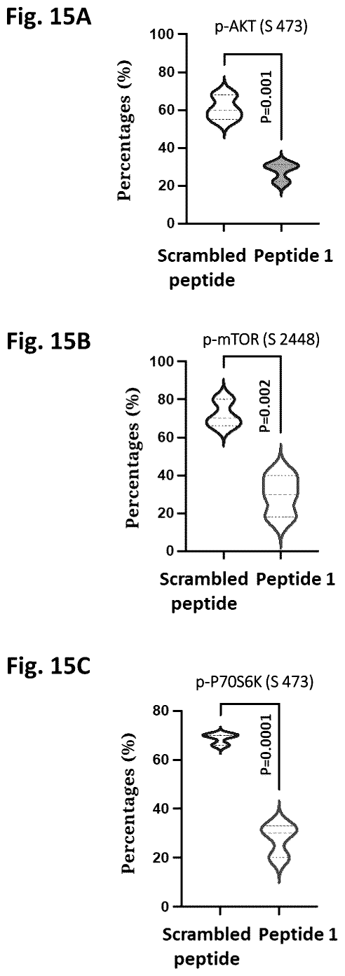

To further confirm/establish the ability of the modulator peptides to attenuate proliferative and/or oncogenic properties of Hep3B HCC cells, the level of activated markers belonging to the Akt- and mTOR-signaling pathways downstream of β-neurexin was evaluated (FIG. 15A-15C). Level of expression of phosphorylated AKT/mTOR/P70S6K was evaluated using flow cytometry in Hep3B cells treated with peptide 1 or with a control peptide having a scrambled sequence. As seen in FIGS. 15A-15C, the percentage of cells expressing phosphorylated AKT/mTOR/P70S6K and/or the level of expression thereof was reduced by 2-fold following treatment with peptide 1 compared to cells treated with scrambled control peptide, suggesting inhibition of proliferative and/or oncogenic capabilities of Hep3B HCC cells.

Advantageously, these results suggest that modulating NLGn4-Nrx1β PPI using peptide 1 can attenuate proliferative and/or oncogenic properties by reducing expression of activated AKT/mTOR/P70S6K, thereby affecting the Akt- and mTOR-signaling pathways in HCC cells.

### Example 8—NLGn4-Nrx1β PPI Peptides Modulate Cell Death of Hepatocellular Carcinoma (HCC) Cells In Vitro

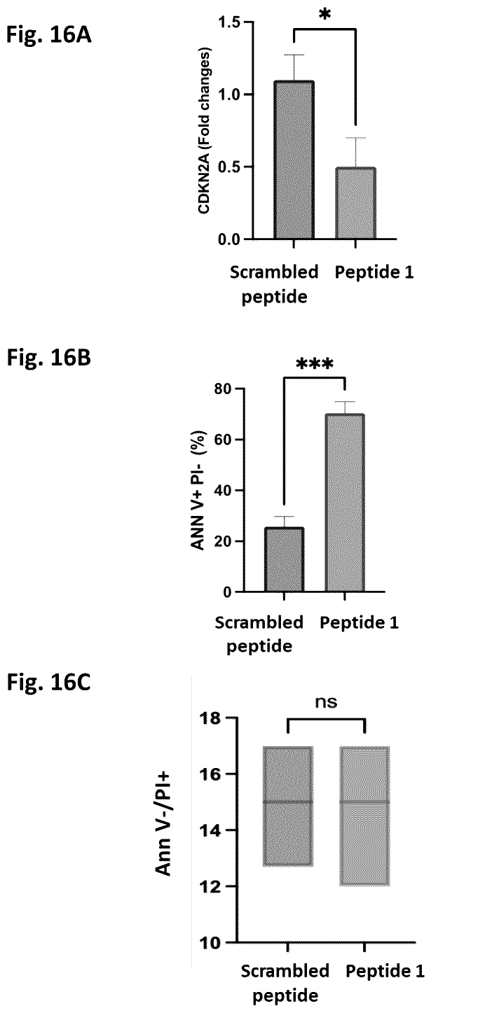

To assess whether the ability of the modulator peptides to attenuate cell division and activation of oncogenic signaling pathways is accompanied with a change in cell death and viability, the effect of inhibiting NLGn4-Nrx1βPPI using peptide 1 was evaluated using markers for viability and cell death, including markers indicative of apoptosis and necrosis (FIG. 16A-16C). HCC Hep3B cells treated with peptide 1 showed 2-fold reduction in the level of expression of the necrotic marker CDNK2A compared with cells treated with control scrambled peptide (FIG. 16A), and 3-fold increase in the level of expression of the apoptotic marker phosphatidylserine which is represented as Annexin-V+/PI− for apoptotic cells compared with cells treated with control scrambled peptide (FIG. 16B), while the viability of non-apoptotic cells represented as Annexin-V−/PI+ remained the same (FIG. 16C).

Advantageously, these results suggest that modulating NLGn4-Nrx1β PPI using peptide 1 can increase the tendency of HCC cells to go through programmed cell death while reducing necrosis and retaining viability of non-apoptotic cells.

### Example 9—Effects of the Modulator Peptides on Various Types of Cancer Cells

The effect of the modulator peptides on various cancer cells, including, effect on cell division, cell death and/or cell viability is evaluated using various markers and/or assays indicative of apoptosis and/or necrosis.

The tested cells include:
Colorectal cancer cells, including Caco2 (cell model, a clone of colorectal adenocarcinoma cells from human).
Breast Cancer cells, including, MCF-7 cells (human breast cancer cell line with estrogen, progesterone, and glucocorticoid receptors).
Prostate cancer cells: PC3 cells (a human androgen-independent prostate cancer cell).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-32. (canceled)

33. An isolated modulator peptide comprising an amino acid sequence as denoted by SEQ ID NO: 6, capable of affecting neuroligin 4 (NLGn4X)-Neurexin 1β (Nrx1β) protein-protein interaction.

34. The isolated modulator peptide according to claim 33, wherein the isolated modulator peptide comprises an amino acid sequence as denoted by any one of SEQ ID NO: 1, SEQ ID NO: 2 and SEQ ID NO: 3.

35. The isolated modulator peptide according to claim 33, consisting of an amino acid sequence as denoted by any one of SEQ ID NO: 1, SEQ ID NO: 2, SEQ ID NO: 3 and/or SEQ ID NO: 6.

36. A composition comprising an isolated modulator peptide according to claim 33, and a pharmaceutically acceptable carrier.

37. The composition according to claim 36, comprising a plurality of isolated modulator peptides.

38. The isolated modulator peptide according to claim 33, or a composition comprising the same, capable of reducing activation of hepatic stellate cells (HSCs).

39. A natural killer (NK) cell harboring the isolated modulator peptide according to claim 33, or a composition comprising the same

40. The NK cell according to claim 39, wherein the NK cell is capable of reducing or inhibiting activity of hepatic stellate cells (HSCs).

41. A composition comprising the NK cell according to claim 39.

42. A method of treating, attenuating and/or preventing progression of a liver disorder in a subject in need thereof, the method comprising administering a therapeutically effective amount of one or more of: the modulator peptide according to claim 33, a composition comprising the modulator peptide, an NK cell harboring the isolated modulator peptide, or a composition comprising the NK cell.

43. The method according to claim 42, wherein the liver disorder is selected from fibrosis, non-alcoholic fatty liver disease (NAFLD), non-alcoholic steatohepatitis (NASH), cirrhosis, hepatitis, liver adenoma, insulin hypersensitivity, liver cancer.

44. The method according to claim 42, wherein the liver disorder is liver fibrosis and/or liver cancer.

45. The method according to claim 42, wherein the cancer is selected from: liver cancer, colorectal cancer, breast cancer and prostate cancer.

46. The method according to claim 42, wherein the administering comprises systemic or localized administration.

47. The method according to claim 46, further comprising administering at least one additional therapeutic agent.
